(19) United States
(12) Patent Application Publication
(10) Pub. No.: US 2020/0246352 A1
(43) Pub. Date: Aug. 6, 2020

(54) Vanadyl and vanadate for use in reducing stress-induced metabolic derangement

(71) Applicant: CFM Pharma Holding B.V., IJsselstein (NL)

(72) Inventors: Hendrik Jan Cornelis Meijerink, Wespelaar (BE); Josephus Johannes de Kimpe, Utrecht (NL); Lekhram Changoer, IJsselstein (NL)

(73) Assignee: CFM Pharma Holding B.V., IJsselstein (NL)

(21) Appl. No.: 16/649,153

(22) PCT Filed: Sep. 21, 2018
(86) PCT No.: PCT/NL2018/050628

§ 371 Date: Mar. 20, 2020

(30) Foreign Application Priority Data
Sep. 22, 2017 (NL) 2019605

(51) Int. Cl. A61K 31/555; A61K 33/24; A61P 3/06

(52) U.S. Cl. CPC A61K 31/555; A61P 3/06; A61K 33/24

(57) ABSTRACT

The current invention relates to the use of a physiologically acceptable organic and/or inorganic vanadium compound or complex, such as for example bis(maltolato)oxovanadium (BMOV) in the prevention or amelioration of stress-induced metabolic derangement in a subject. More in particular, the invention relates to a physiologically acceptable organic and/or inorganic vanadium compound or complex for use in the amelioration of hyperglycemia in a subject suffering from stress such as elicited by a trauma, wherein the physiologically acceptable organic and/or inorganic vanadium compound or complex is administered to said subject before the trauma is inflicted to the subject. Furthermore, the invention relates to a physiologically acceptable organic and/or inorganic vanadium compound or complex for use in the prevention of hyperglycemia in a subject having a trauma, wherein the physiologically acceptable organic and/or inorganic vanadium compound or complex is administered to said subject before the subject has the trauma. In one embodiment a physiologically acceptable organic and/or inorganic vanadium compound or complex is administered to a human subject 2 to 24 hours before said human subject is subjected to surgery, for the prevention or amelioration of hyperglycemia elicited by a trauma related to the surgery. It is part of the invention that the physiologically acceptable organic and/or inorganic vanadium compound or complex are a source of vanadyl or vanadate in a patient to whom such compound or complex is administered.

### FIELD OF THE INVENTION

The current invention relates to the use of a physiologically acceptable organic and/or inorganic vanadium compound or complex such as bis(maltolato)oxidovanadium (BMOV) in the prevention or amelioration of stress-induced metabolic derangement in a subject. More in particular, the invention relates to a physiologically acceptable organic and/or inorganic vanadium compound or complex for use in the amelioration of hyperglycemia in a subject suffering from stress such as elicited by a trauma, wherein the physiologically acceptable organic and/or inorganic vanadium compound or complex is administered to said subject before the trauma is inflicted to the subject. Furthermore, the invention relates to a physiologically acceptable organic and/or inorganic vanadium compound or complex for use in the prevention of hyperglycemia in a subject having a trauma, wherein the physiologically acceptable organic and/or inorganic vanadium compound or complex is administered to said subject before the subject has the trauma. In one embodiment the physiologically acceptable organic and/or inorganic vanadium compound or complex is administered to a human subject 0 to 96 hours before said human subject is subjected to surgery, preferably 2 to 24 hours, for the prevention or amelioration of hyperglycemia elicited by a trauma related to the surgery. It is part of the invention that the physiologically acceptable organic and/or inorganic vanadium compound or complex are a source of vanadyl or vanadate in a patient to whom such compound or complex is administered.

### BACKGROUND OF THE INVENTION

Metabolic derangement is an important and common hallmark of stress exposed to a subject. Examples of such stress inducing metabolic derangement in a subject is stress related to traumatic injury, exposure of a body to irradiation such as radiation therapy in the treatment of cancer, and administering contrast fluid or agent to a subject, such as for the purpose of imaging of (parts of) the body. When for example a trauma is inflicted in a subject such as a human subject, metabolic aberrations in plasma are identified, which aberrations are triggered by the trauma and for example are further triggered by shock such as hemorrhagic shock. Alteration in metabolites associated with catabolism, acidosis and hyperglycemia have been identified. For example, trauma can trigger severe metabolic derangement, resulting in increased glucose levels, ultimately resulting in amongst others hyperglycemia in plasma of a subject experiencing such stressful event. Conventional measures of biochemical imbalance following stress such as stress related to a trauma have been shown to correlate with patient outcome. Catabolism, acidosis, and insulin resistance with resultant hyperglycemia are examples of defined metabolic phenotypes (metabotypes) contributing to secondary injury following trauma.

Stress hyperglycemia (also called stress diabetes or diabetes of injury) refers to transient elevation of the blood glucose due to the stress of an illness or of a trauma. Such hyperglycemia is a significant problem in patients suffering from a trauma or in patients subjected to for example surgery which inflicts a trauma in the patient, radiation therapy, acquiring an infection, administration of a medicine, psychical stress. Hyperglycemia has been shown to be associated with increased morbidity and mortality. As said, hyperglycemia in for example the trauma patient is caused by a hypermetabolic response to stress. Stress-induced hyperglycemia is at the basis of numerous health-threatening effects in the patient suffering from the increased blood glucose level. Hyperglycemia is involved for example in: exacerbation of both calcium imbalance and the accumulation of reactive oxygen species (ROS) in neurons, leading to increased apoptosis; fueling anaerobic energy production, causing lactic acidosis, which further stresses neurons in the penumbral regions; decreasing blood perfusion after ischemic stroke by lowering the availability of nitric oxide (NO), which is a crucial mediator of vasodilation; and intensifying the inflammatory response after stroke, causing edema, and hemorrhage through disruption of the blood brain barrier and degradation of white matter, which leads to a worsening of functional outcomes.

Surgical patients commonly develop hyperglycemia related to the hypermetabolic stress response, which increases glucose production and causes insulin resistance. Although hyperglycemia is associated with worse outcomes, the treatment of hyperglycemia with insulin infusions has not provided consistent benefits. Despite first results, which suggested decreased mortality and other advantages of a relatively tight glucose control, further investigations identified no benefit or increased mortality when hyperglycemia was aggressively treated with insulin. Because of these conflicting data, the optimal glucose concentration to improve outcomes is unknown. There is agreement, however, that hypoglycemia is an undesirable complication of intensive insulin therapy and should be avoided. In addition, the risk of increased glucose variability is recognized, because of the associated increased risk for worse outcomes.

Stress hyperglycemia is especially common in patients with hypertonic dehydration, in patients with elevated catecholamine levels (e.g., after emergency department treatment of acute asthma with epinephrine), in patients with increased hormone levels like corticosteroids or growth hormone, and in patients with blood loss, increased heart rate and/or low blood pressure following trauma or physical or infectious insult—but there are many more examples e.g. ischemic stress such as stroke. Stress hyperglycemia increases the risk for postoperative infectious complications after surgery. For example, a subject suffering from an acute myocardial infarction can present with a blood glucose level of over 180 mg/dL, which high level is associated with increased risk of congestive cardiac failure or cardiogenic shock. Furthermore, acute stroke, that is to say the acute phase of stroke, is accompanied with hyperglycemia, which hyperglycemia has been established as a predictor of poor outcome in non-diabetic patients. It has also been revealed that for in-hospital patients, hyperglycemia was not only an independent marker of in-hospital mortality in intensive care units but also in patients admitted to general hospital wards. Total mortality is significantly higher in patients with such hyperglycemia (16%) than in diabetic patients (3%) and normoglycemic patients (1.7%).

People who have experienced stress hyperglycemia during severe illness have a threefold risk of developing diabetes in subsequent years, and it may be appropriate to screen for diabetes in survivors of critical illness.

From the above outlined issues with regard to the occurrence of hyperglycemia in subjects exposed to stress such as the surgical patients, and with regard to the risk for subsequent occurrence of hypoglycemia due to too tight glucose level management in the surgical patient, it is clear that current standard of care with regard to treatment of hyperglycemia in the patient in which trauma is inflicted, is far from optimal. The more since the current standard of care is centered around management of glucose level in the blood of subjects while such subjects are being subjected to the stress eliciting the metabolic derangement, or shortly thereafter, thus wherein the management of glucose level in the blood in most occasions relates to the treatment of existing hyperglycemia.

Therefore, a high need exists for developing treatment modalities that limit metabolic derangements in patients to which stress is elicited such as patients subjected to surgery inflicting trauma to the patient, so as to design treatment strategies tailored toward metabolic alterations and the severity of stress- and trauma-induced treats to the patient's health. In particular, the devastating consequences of hyperglycemia as a result of trauma, requires high attention, and thus, in particular a solution to the problem of hyperglycemia in patients subjected to trauma, such as trauma related to surgery, is highly needed.

### SUMMARY OF THE INVENTION

Current standard of care for management of hyperglycemia related to stress-induced metabolic derangement in a patient is focused on lowering blood glucose levels in the patient after the patient started to be exposed to stress such as stress related to an illness or related to trauma during surgical measures. Treatment of already stress-inflicted hyperglycemia then bears the risk of damage already caused in the patient by the hyperglycemic state before glucose levels are reduced to normal in the blood of the patient, and/or bears the risk of being too harsh such that glucose levels are lowered to an extent that hypoglycemia is induced.

A first aspect of the current invention relates to a pharmaceutical composition comprising a physiologically acceptable organic and/or inorganic vanadium compound or complex and further comprising a pharmaceutically acceptable excipient for use in the prevention or reduction of stress-induced metabolic derangement in a patient subjected to stress. The stress elicits a state of physiological strain in the patient, and the stress is of physical nature (e.g. trauma, infection, radiation therapy) and/or of psychical origin (e.g. acute anxiety, exposure to a future threat to the subject's body- or mental integrity). Preferably, the physiologically acceptable organic and/or inorganic vanadium compound or complex is an insulin-mimetic vanadium compound. Preferably, the physiologically acceptable organic and/or inorganic vanadium compound or complex is bis(maltolato)oxidovanadium (BMOV). Also according to the invention, the physiologically acceptable organic and/or inorganic vanadium compound or complex for use of the invention is a compound or complex capable of providing a vanadate moiety or a vanadyl moiety in the body of a subject such as a human subject, once the compound or complex is administered to said subject.

It is now due to the contribution of the current inventors that a subject, such as a human subject, can be subjected to preventive measures, i.e. administering a pharmaceutical composition comprising a physiologically acceptable organic and/or inorganic vanadium compound or complex before the subject is exposed to stress, such that glucose levels in the blood of the subject do not rise to a high extent while the subject is exposed to stress, and such that hyperglycemia does not occur.

In one embodiment, the pharmaceutical composition is administered to a patient before the patient is subjected to a stress.

In one embodiment, the pharmaceutical composition for use according to the invention is for use in the prevention or reduction of stress-induced metabolic derangement in a patient subjected to stress, wherein the metabolic derangement comprises hyperglycemia, preferably the metabolic derangement is hyperglycemia.

Trauma related to surgery is a typical type of stress that can inflict hyperglycemia in the patient, said hyperglycemia causing damage to the patient, sometimes even irreversible damage. Therefore, in one embodiment, the pharmaceutical composition is administered to a patient before the patient is exposed to stress such as being subjected to surgery. Further types of stress are for example a trauma caused by a medical procedure, a trauma caused by iatrogenic injury, and/or a stress caused by any one or more of an infection, administration of one or more medicine(s), either long term or short term acute mental stress due to a life threatening situation, such as a potentially life threatening situation and an accident, according to the invention.

Typically, according to the invention, the pharmaceutical composition comprising a physiologically acceptable organic and/or inorganic vanadium compound or complex such as BMOV for use according to the invention is administered to a subject 0 h to 96 h before e.g. surgery, for example administered twice at about 16 h to 24 h, or about 16 h to 20 h, before surgery and a second time at about 3 h before said surgery.

In one embodiment, the pharmaceutical composition comprising a physiologically acceptable organic and/or inorganic vanadium compound or complex such as BMOV for use according to the invention is administered orally or parenterally, e.g. intravenously or intraperitoneally or subcutaneously or intramuscularly or intradermally to a subject who is about to be subjected to a stress, such as a subject who will undergo surgery.

In one embodiment, the pharmaceutical composition comprising a physiologically acceptable organic and/or inorganic vanadium compound or complex such as BMOV for use according to the invention, is administered to a patient such that the increase of glucose content in the blood of said patient during the first one to eight hours, preferably during the first about three hours of the period in which the patient is subjected to a stress is between about −25% and about 100%, preferably between about 0% and about 75%, more preferably between about 5% and about 65%, most preferably about 30%, compared to the glucose content in the blood of said patient at a time point 3 h to 0 minute before the patient is subjected to the stress, preferably compared to the glucose content in the blood of the patient at the time point at which the patient starts to be subjected to the stress.

A second aspect of the current invention is a kit of parts comprising a container comprising at least two doses of the pharmaceutical composition comprising a physiologically acceptable organic and/or inorganic vanadium compound or complex such as BMOV for use according to the invention, the kit further comprising instructions for use of the pharmaceutical composition comprising a physiologically acceptable organic and/or inorganic vanadium compound or complex such as BMOV provided in said containers, in the prevention or reduction of stress-induced metabolic derangement in a patient who is to be subjected to stress which stress elicits a state of physiological strain in the patient.

As said, it is also part of the invention that the physiologically acceptable organic and/or inorganic vanadium compound or complex are a source of vanadyl or vanadate in a patient to whom such compound or complex is administered when used in the prevention or reduction of stress-induced metabolic derangement in a patient subjected to stress.

### Definitions

The term “stress” has its regular scientific meaning such as outlined in the Dorland's Pocket Medical Dictionary, 26^{th} Ed., and here refers to an adverse physical and/or psychical stimulus, internal or external, that elicits a stress reaction and/or a state of physiological strain disturbing the functioning of an organism, here a subject such as a human subject.

The term “metabolic derangement” has its normal scientific meaning and here refers to amongst others hyperglycemia, lactic acidosis and hyperlipidemia.

The term “hyperglycemia” regular scientific meaning such as outlined in the Dorland's Pocket Medical Dictionary, 26^{th} Ed., and here refers to an abnormally increased content of glucose in the blood.

The term “trauma” has its normal scientific meaning and here refers to an injury to a subject's body, such as a human subject. Typically, an injury relates to damage to the subject's body originating from for example an infection, surgery, an accident, internal damage caused by stroke and/or ischemia, internal conditions leading to acute organ failure.

The term ‘excipient’ as used herein has its conventional meaning and refers to a pharmaceutically acceptable ingredient, which is commonly used in the pharmaceutical technology for preparing a formulation such as a dosage formulation, for example for oral administration, intradermal administration, intravenous administration.

The term ‘pharmaceutical composition’ as used herein has its conventional meaning and refers to a composition which is pharmaceutically acceptable.

The term ‘pharmaceutically acceptable’ as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio.

‘Bis(maltolato)oxovanadium’, also referred to as ‘bis(maltolato)oxovanadium(IV)’ and ‘bis(maltolato)oxo-vanadium’ and ‘bis(maltolato)oxidovanadium’, ‘BMOV’ in short, refers to the vanadium(IV) complex having a molecular structure as outlined in FIG. 7.

‘Bis(ethylmaltolato)oxovanadium(IV)’ and ‘bis(ethylmaltolato)oxidovanadium(IV)’ and ‘bis(ethylmaltolato)oxidovanadium’, ‘BEOV’ in short, refers to the vanadium(IV) complex having a molecular structure as outlined in FIG. 8.

### BRIEF DESCRIPTION OF THE DRAWINGS

FIG. 1. Experimental design for adrenaline/saline and BMOV/vehicle administration as well as for blood sampling. BMOV dose 1 started 120 minutes after the onset of adrenaline infusion or of saline infusion.

FIG. 2. Blood glucose level (mean+/−SEM) after administration of low, medium and high dose of adrenaline infusion (n=4-5/group).

FIG. 3. Blood glucose level (mean+/−SEM) in series 2 (n=1-2 rats/group).

FIG. 4. Experimental design applied for adrenaline administration and BMOV administration as well as for blood sampling. Dose of adrenalin infusion was: 0.3 microgram/kg/min.

FIG. 5. Pretreating subjects with BMOV (3 h before occurrence of stress, or at 16-24 h and subsequently at 3 h before occurrence of the stress) before occurrence of a stressful insult (here, the infusion of adrenaline starting at t=0 minutes) results in a surprisingly high reduction of the occurrence of hyperglycemia in the subjects suffering from stress.

FIG. 6. Pretreating subjects with BMOV (3 h before occurrence of stress, or at 16-24 h and subsequently at 3 h before occurrence of the stress) before occurrence of a stressful insult (here, the infusion of adrenaline starting at t=0 minutes) results in a surprisingly high reduction of the occurrence of hyperglycemia in the subjects suffering from stress. Here, the glucose level in the blood of the subject exposed to the stress is shown at the time point 180 minutes after the start of the adrenaline infusion.

FIG. 7. Molecular structure of bis(maltolato)oxovanadium (BMOV).

FIG. 8. Molecular structure of bis(ethylmaltolato)oxovanadium(IV) (BEOV).

### DETAILED DESCRIPTION OF THE INVENTION

The present invention will be described with respect to particular embodiments and with reference to certain examples but the invention is not limited thereto but only by the claims.

Furthermore, the various embodiments, although referred to as “preferred” are to be construed as exemplary manners in which the invention may be implemented rather than as limiting the scope of the invention.

The term “comprising”, used in the claims, should not be interpreted as being restricted to the elements or steps listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a composition comprising A and B” should not be limited to compositions consisting only of components A and B, rather with respect to the present invention, the only enumerated components of the composition are A and B, and further the claim should be interpreted as including equivalents of those components, such as derivatives thereof.

As used herein, the term “may” encompasses the word “can,” and the term “may be” encompasses the words “is” or “are,” depending on context. Furthermore, presence of the word “may” is intended to explain options for practicing or implementing the disclosure, without limitation.

Besides the above mentioned physiologically acceptable organic and/or inorganic vanadium compound or complex such as a vanadium-based insulin-mimetic or such as BMOV, the pharmaceutical composition according to the present invention also comprises a pharmaceutically acceptable excipient.

For the purpose of oral administration of the pharmaceutical composition of the invention, such an excipient is chosen from ingredients which are commonly used in the pharmaceutical technology for preparing granulate, solid or liquid oral dosage formulations. Similarly, such an excipient is chosen from ingredients which are commonly used in the pharmaceutical technology for preparing formulations for intravenous administration, intramuscular administration, etc.

Examples of categories of excipients include, but are not limited to, binders, disintegrants, lubricants, glidants, fillers and diluents. For oral administration of the pharmaceutical composition of the invention, one of ordinary skill in the art may select one or more of the aforementioned excipients with respect to the particular desired properties of the granulate e.g. from which tablets or pills are produced by routine experimentation and without any undue burden. The amount of each excipient used may vary within ranges conventional in the art. The following references which are all hereby incorporated by reference disclose techniques and excipients used to formulate pharmaceutical compositions, such as pharmaceutical compositions for oral administration. See “The Handbook of Pharmaceutical Excipients”, 4th edition, Rowe et al., Eds., American Pharmaceuticals Association (2003); and “Remington: The Science and Practice of Pharmacy”, 20th edition, Gennaro, Ed., Lippincott Williams & Wilkins (2000).

A first aspect of the invention relates to a pharmaceutical composition comprising a physiologically acceptable organic and/or inorganic vanadium compound or complex and further comprising a pharmaceutically acceptable excipient for use in the prevention or reduction of stress-induced metabolic derangement in a patient subjected to stress.

An aspect of the invention relates to a pharmaceutical composition comprising a physiologically acceptable organic and/or inorganic vanadium compound or complex and further comprising a pharmaceutically acceptable excipient for use in the prevention or reduction of stress-induced metabolic derangement in a patient subjected to stress, wherein the physiologically acceptable organic and/or inorganic vanadium compound or complex for use of the invention is a compound or complex capable of providing a vanadate moiety or a vanadyl moiety in the body of a subject such as a human subject, once the compound or complex is administered to said subject. Thus, according to the invention, the pharmaceutical composition comprising a physiologically acceptable organic and/or inorganic vanadium compound or complex for use according to the invention is a pharmaceutical composition that is a source of vanadyl or vanadate in the body of the subject to whom the pharmaceutical composition is administered. According to the invention, an amount of a pharmaceutical composition comprising a physiologically acceptable organic and/or inorganic vanadium compound or complex for use according to the invention is administered to a subject such that said amount provides for a plasma exposure level of between 25 ng/ml and 2500 ng/ml elemental vanadium or more preferably between 100 ng/ml and 1000 ng/ml in said subject.

In one embodiment, the pharmaceutical composition for use according to the invention is administered to the patient before the patient is subjected to the stress. In many occasions, it is known at what day and time a subject such as a human subject will be subjected to stress such as stress inflicted by trauma related to surgery. Such circumstances of knowledge about when which trauma will be inflicted to a subject, provides the opportunity to benefit from the current invention. That is to say, a subject who will be subjected to for example surgery, is administered at least one dose, preferably two doses of a pharmaceutical composition comprising BMOV at a time point before the stress is elicited to the subject. Then, this way, the subject is prevented from the occurrence of metabolic derangement during the time period in which stress is elicited, e.g. during surgery, and in particular the subject undergoing surgery is prevented from suffering from hyperglycemia, according to the invention. As said before, preventing or diminishing a rise in glucose level in the blood improves mortality and morbidity, which is thus one of the many benefits of the current invention.

An aspect of the present invention relates thus to a pharmaceutical composition comprising a physiologically acceptable organic and/or inorganic vanadium compound or complex and further comprising a pharmaceutically acceptable excipient for use in the prevention or reduction of stress-induced metabolic derangement in a patient subjected to stress, wherein the pharmaceutical composition is administered to the patient before the patient is subjected to the stress. According to the invention, the vanadium compound is an insulin mimetic and preferably the vanadium compound is selected from BMOV and BEOV, preferably BMOV. Also according to the invention, the physiologically acceptable organic and/or inorganic vanadium compound or complex is a compound or complex capable of providing a vanadate moiety or a vanadyl moiety in the body of a subject such as a human subject, once the compound or complex is administered to said subject, e.g. a non-diabetic human subject. According to the invention, the physiologically acceptable vanadium compound or vanadium complex is preferably a compound or complex comprising a vanadium cation with a valence of +4 or +5, i.e. V⁴⁺ or V⁵⁺. Preferably, the vanadate moiety of the vanadium complex or compound is a V⁴⁺ cation or a V⁵⁻ cation, preferably V⁵⁺. Preferably, the vanadyl moiety of the vanadium complex or compound is a V⁴⁺ cation.

According to the invention, the physiologically acceptable organic and/or inorganic vanadium compound or complex comprised by the pharmaceutical composition for use according to the invention, is a compound or complex selected from the group of vanadium complexes and vanadium compounds comprising: BMOV, BEOV, an insulin mimetic, vanadyl acetylacetonate (VAC), an organo-vanadium compound that has demonstrated insulin-mimetic effects in type 1 and/or type 2 diabetes mellitus in a subject such as a human, an organo-vanadium compound that has a pharmacological activity selected from inhibition of gluconeogenesis, a decrease in glutamate dehydrogenase activity, and antilipolysis; vanadyl sulfate (VS), vanadyl 3-ethylacetyl-acetonate (VET); oxovanadium, orthovanadium; a vanadium (IV) coordinate covalently bound to an organic moiety selected from the group consisting of keto-enol tautomers with the keto and enol groups on adjacent carbon atoms that form 5-membered rings including the metal, and beta diketones in which the two keto groups are separated by one carbon atom, that form a 6-membered ring including the metal, preferably a keto-enol tautomer, preferably the organic moiety is selected from the group consisting of maltol, 2-hydroxy-2,4,6-cycloheptatrien-1-one, 3-bromo-2-hydroxy-2,4,6-cycloheptatrien-1-one, 2-hydroxy-4-isopropyl-2,4,6-cycloheptatrien-1-one, 2-hydroxy-4-methyl-2,4,6-cycloheptatrien-1-one, 3-hydroxy-1,2-dimethyl-4(1H)-pyridone, 3-ethyl-2-hydroxy-2-cyclopenten-1-one, 3,4-dihydroxy-3-cyclobuten-1,2-dione, ethyl 2-hydroxy-4-oxo-2-pentenone, 2,3,5,6-tetrahydroxy-1,4-benzoquinone, 2′,4′-dihydroxy-2-methoxyacetophenone, 4-hydroxy-5-methyl-4-cyclopenten-1,3-dione, 2-chloro-3-hydroxy-1,4-naphthoquinone, 2-(4-bromophenyl)-3-hydroxymaleimide, 2-hydroxy-3-methyl-2-cyclopenten-1-one, 2′,3′,4′-trihydroxyacetophenone, furoin, 2-hydroxy-2-methylpropiophenone, maclurin, 6-(pyrrolidinomethyl) kojic acid, alpha-acetyl-4-hydroxy-beta-(hydroxymethyl)-3-methoxycinnamic acid gamma-lactone, 4-hydroxy-5-phenyl-4-cyclopenten-1,3-dione, 6-(morpholinomethyl) kojic acid, 1-(4,5-dimethoxy-2-hydroxyphenyl)-3-methyl-2-buten-1-one, purpurogallin, 2,3-dihydroxy-1,4-phenazinedione, alizarin orange, 1-hydroxy-1-methylnaphthalen-2(1H)-one, alizarin, 6-(piperidinomethyl) kojic acid, 1,2,7-trihydroxyanthraquinone, 6-(4-methylpiperazinomethyl) kojic acid, fisetin, 3-oxo-4,5,6-trihydroxy-3(H)-xanthene-9-propionic acid, benzoin, 4′-chlorobenzoin, quercetin, morin, myricetin, and 4,4′-dimethylbenzoin, more preferably the organic moiety is maltol or beta diketone, the organic moiety is selected from the group consisting of acetylacetone, 2-acetyl-1-tetralone, benzoylacetone, 1-benzoylacetylacetone, 1,1,1-trifluoro-2,4-pentanedione, S-methyl-4,4,4-trifluoro-3-oxothiobutyrate, 2-acetyl-1,3-cyclopentanedione, 3-chloro-2,4-pentanedione, 1,1,1,5,55,-hexafluoro-2,4-pentanedione, 3-ureidomethylene-2,4-pentanedione, 2-acetylcyclopentanone, 2-acetylcyclohexanone, 3-methyl-2,4-pentanedione, 2,4,6-heptatrione, 3-ethyl-2,4-pentanedione, thianoyltrifluoroacetone, S-t-butyl-acetothioacetate, 3-acetyl-5-methylhexan-2-one, 3-acetyl-2-heptanone, 2,2-dimethyl-6,6,7,7,8,8,8-heptafluoro-3,5-octanedione, 4-hydroxy-5-phenyl-4-clyclopenten-1,3-dione, 4,4,4-trifluoro-1-phenyl-1,3-butanedione, 3-acetyl-2-octanone, 1(2-hydroxy-4-methylphenyl)-1,3-butanedione, 1-(2-hydroxy-5-methylphenyl)-1,3-butanedione, 3-benzylidene-2,4-pentanedione, 1-(2-hydroxy-5-methylphenyl)-1,3-pentanedione, 2,2,6,6-tetramethyl-3,5-heptanedione, 3-acetyl-5-hydroxy-2-methylchromone, (+)-3-(trifluoroacetyl)camphor, 4,9-dihydro-6-methyl-5H-furo(3,2-g) (1) benzopyran-4,5,9-trione, 3-(2-nitrobenzylidene)-2,4-pentanedione, 1,3-bis-(4-chlorophenyl)-1,3-propanedione, 1,3-bis-(4-fluorophenyl)-1,3-propanedione, 4,4,4-trifluoro-1-(2-naphthyl)-1,3-butanedione, 1-(2-hydroxyphenyl)-3-(4-methoxyphenyl)-1,3-propanedione, 2-bromo-1,3-diphenyl-1,3-propanedione, dibenzoylmethane, 2-(4-chlorobenzylidene)-1-phenyl-1,3-butanedione, 2-(2-nitrobenzylidene)-1-phenyl-1,3-butanedione, bis(4-methoxybenzoyl) methane, and curcumin, preferably the organic moiety is 2-acetyl 1-tetralone; a coordinate-covalent complex including a vanadium (V) metal ion, an oxo group coordinate-covalently bound to the metal ion, two peroxo groups coordinate-covalently bound to the metal ion, and at least one organic moiety coordinate-covalently bound to the metal ion through at least one N-containing or O-containing functional group capable of donating electrons through a coordinate-covalent bond, wherein the coordinate-covalent complex is selected from the group consisting of (1,10-phenanthroline) (oxodiperoxovanadium(V), oxalato-oxodiperoxovanadium(V), (2,2′-bipyridine) oxodiperoxovanadium(V), (4,7-dimethyl-1,10-phenanthroline) oxodiperoxovanadium(V), (3,4,7,8-tetramethyl-1,10-phenanthroline) oxodiperoxovanadium(V), (pyridine-2-carboxylic acid) oxodiperoxovanadium(V), (5-hydroxypyridine-2-carboxylic acid) oxodiperoxovanadium(V), (pyridine-2,6-dicarboxylic acid) oxodiperoxovanadium(V), and derivatives thereof, preferably the coordinate-covalent complex is (1,10-phenanthroline)oxodiperoxovanadium (V); a coordinate-covalent complex including a vanadium(V) metal ion, an oxo group coordinate-covalently bound to the metal ion, one peroxo group coordinate-covalently bound to the metal ion, and at least one organic moiety coordinate-covalently bound to the metal ion through at least one N-containing or O-containing functional group capable of donating electrons through a coordinate-covalent bond wherein the coordinate-covalent complex is (pyridine-2,6-dicarboxylato) (hydrato) oxoperoxovanadium(V); a coordinate-covalent complex selected from the group consisting of (1,10-phenanthroline)oxodiperoxovanadium (IV), oxalatooxodiperoxovanadium (V), (2,2′-bipyridine) oxodiperoxovanadium (V), (4,7-dimethyl-1,10-phenanthroline) oxodiperoxovanadium (V), (3,4,7,8-tetramethyl-1,10-phenanthroline) oxodiperoxovanadium (V), (pyridine-2-carboxylic acid) oxodiperoxovanadium (V), (5-hydroxvpyridine-2-carboxylic acid) oxodiperoxovanadium (V), (pyridine-2,6-dicarboxylic acid) oxodiperoxovanadium (V), (pyridine-2,6-dicarboxylato) (hydrato) oxoperoxovanadium (V), and derivatives thereof substituted with hydroxy or lower alkyl substituents that do not interfere with the formation of coordinate-covalent bonds; a coordinate-covalent complex including, a vanadium(V) metal ion, an oxo group coordinate-covalently bound to the metal ion, and at least one organic moiety coordinate-covalently bound to the metal ion through at least one N-containing or O-containing moiety capable of donating electrons to a coordinate-covalent bond wherein the coordinate-covalent complex is meso-tartrato oxovanadium(V); peroxido vanadium compounds with picolinate and phenanthroline, preferably bis peroxovanadium-picolinate (bpV(pic), bpV(HOpic), bpV(phen), VO(OPT). Preferred are physiologically acceptable vanadium complexes or vanadium compounds capable of releasing or forming vanadyl and/or vanadate and/or bis-peroxovanadate, according to the invention. Without wishing to be bound by theory, the active moieties of the different vanadium compounds in the subject to whom the pharmaceutical composition is administered according to the use of the invention, are vanadyl (VO²⁺) or vanadate (H₂VO₄⁻). Therefore, a vanadium complex or vanadium compound for use in a pharmaceutical composition according to the invention is any physiologically acceptable vanadium complex or vanadium compound that provides such a vanadyl (VO²⁺) or vanadate (H₂VO₄⁻) in the subject once the pharmaceutical composition comprising said vanadium compound or complex is administered according to the use of the invention to a subject.

In one embodiment, the pharmaceutical composition for use according to the invention is administered to a subject wherein the subject is an animal such as a mammal, preferably the subject is a human, such as a healthy human, a non-diabetic human and a diabetic human, preferably a non-diabetic human, such as a healthy non-diabetic human.

A typical a pharmaceutical composition of the invention is a pharmaceutical composition comprising bis(maltolato)oxidovanadium (BMOV) or comprising bis(ethylmaltolato)oxovanadium(IV) (BEOV), preferably BMOV or comprising a combination thereof. Preferably, the physiologically acceptable organic and/or inorganic vanadium compound or complex is a vanadium compound such as a vanadium-based insulin-mimetic.

The stress to which the subject is exposed or subjected, elicits a state of physiological strain in the patient, and the stress is of physical nature (e.g. trauma, infection, radiation therapy) and/or of psychical origin (e.g. acute anxiety, exposure to a future threat to the subject's body- or mental integrity). In one embodiment, the pharmaceutical composition for use according to the invention comprises a physiologically acceptable organic and/or inorganic vanadium compound or complex wherein said vanadium compound or complex is bis(maltolato)oxidovanadium (BMOV) or bis(ethylmaltolato)oxovanadium(IV) (BEOV), preferably BMOV.

In one embodiment, the pharmaceutical composition for use according to the invention is for use in the prevention or reduction of stress-induced metabolic derangement in a patient subjected to a stress, wherein the metabolic derangement comprises hyperglycemia, preferably the metabolic derangement is hyperglycemia.

Stress induced metabolic derangement such as hyperglycemia can be caused by a multiple of events and occasions. In physical medicine, major trauma is injury or damage to a biological organism caused by physical harm from an external source. Major trauma is also injury that can potentially lead to serious long-term outcomes like chronic pain. Major trauma is an example of stress that induces hyperglycemia.

Surgery is a major stress inducing hyperglycemia. Non-limiting examples of surgical measures and diseases and health issues that are examples of acute stress inducing hyperglycemia, are: myocardial infarction (MI), stroke, multiple trauma, burn wounds, decubitus, tracheal intubation, glaucoma and other eye surgery (laser, eye corrections), anesthesia, cosmetic surgery, liposuction, sepsis, shock, hemorrhage, acute kidney injury, acute intestinal ischemic injury, traumatic brain injury (TBI).

A traumatic injury is an injury caused by external force, but which does not rise to the level of major trauma. Traumatic injuries are distinguished from other causes of injury, such as iatrogenic injuries (see below).

Iatrogenesis refers to any effect on a person, resulting from any activity of one or more persons acting as healthcare professionals or promoting products or services as beneficial to health, that does not support a goal of the person affected. Some iatrogenic effects are clearly defined and easily recognized, such as a complication following a surgical procedure (e.g., lymphedema as a result of breast cancer surgery). Less obvious ones, such as complex drug interactions, may require significant investigation to identify. While some have advocated using ‘iatrogenesis’ to refer to all ‘events caused by the health care delivery team’, whether ‘positive or negative’, consensus limits use of ‘iatrogenesis’ to adverse, or, most broadly, to unintended outcomes. Thus, iatrogenesis can be iatrogenic injury, which is a stress able to induce hyperglycemia in a patient.

Causes of iatrogenesis include side effects of possible drug interactions, adverse effects of prescription drugs, complications arising from a procedure or treatment, medical error such as over-use of drugs, a wrong prescription, nosocomial infections, faulty procedures, faulty techniques, faulty information, faulty methods, or faulty equipment.

Acute psychical stress is a further cause of hyperglycemia. Acute anxiety or exposure to circumstances dangerous to the body or even to life are examples of acute psychical stress known to be able to induce hyperglycemia.

Furthermore, postoperative infectious complications is a stress that induces hyperglycemia, coming with a risk of morbidity and mortality.

The current inventors surprisingly found that BMOV bears the potential to prevent occurrence of hyperglycemia in a subject to whom a stress is elicited, when the BMOV is administered to the subject before the subject is exposed to the stress. Prevention of the build-up of a hyperglycemic state in a subject exposed to stress encompasses both the maintenance of normoglycemia in the subject and the inhibition of the rise in glucose level in the blood of the subject to levels designated as hyperglycemia. Furthermore, upon pretreatment of a subject before exposure of the subject to stress with BMOV, prevention of hyperglycemia due to the exposure to stress may also result in a decrease of the initial glucose level in the blood of the subject before the stress was elicited to the subject, for example at the start of the time period in which the subject is exposed to the stress. Normoglycemic values are glucose levels in the blood of between 70 mg/dl and 100 mg/dl in the fasting state (preprandial) of a healthy human subject (4 mmol/l to 6 mmol/l), and glucose levels in the blood of less than 140 mg/dl at a time point 2 hours after a meal (postprandial state of a healthy subject) (less than 7.8 mmol/l). Normal healthy subjects (mammals, here rats) exposed to stress without having received a pretreatment with BMOV experienced an increase in the glucose level in the blood of on average about 260%, in test series executed by the inventors. In contrast, subjects who received treatment regimen comprising administration of two doses of BMOV at different time points before stress was induced only experienced a rise in blood glucose level of at most about +28%, i.e. the maximum average glucose level in the blood was about 6.4 mmol/l. Thus, although subjects are subjected to stress conditions, e.g. stress elicited upon injection of adrenaline, the pretreatment of the subjects with BMOV prevented the build up of hyperglycemia and strongly reduced the increase in glucose level in the blood to a large extent, according to the invention. Introduction of adrenaline in an organism such as a subject, e.g. a human subject, is known for a long time as a stress that causes hyperglycemia. See for example in [“Adrenergic mechanisms of catecholamine action on glucose homeostasis in man.” by Rizza R A, Cryer P E, Haymond M W, Gerich J E., in Metabolism. 1980 November; 29(11 Suppl 1):1155-63].

Reference is made to the examples outlined in the Examples section, below.

In one embodiment, the pharmaceutical composition for use according to the invention comprises as the sole active pharmaceutical ingredient BMOV or BEOV, or comprises as the active pharmaceutical ingredients a combination of BMOV and BEOV. Preferred is a pharmaceutical composition of the invention comprising as the sole active pharmaceutical ingredient BMOV, according to the invention. The current inventors surprisingly found that administering an organic vanadium compound, i.e. BMOV to a subject before the subject is exposed to a stress, such as a rise in adrenaline levels in the circulation, results in prevention or lowering of a rise in glucose level in the blood of the subject during the time period in which the subject is suffering from the particular stress. Thus, by administering a pharmaceutical composition consisting of at least one pharmaceutically acceptable excipient and BMOV to a subject before subjecting the subject to a condition of stress, hyperglycemia is adequately and sufficiently prevented or the extent of a rise in blood glucose level during the period of stress is lowered to a large extent, according to the invention.

In one embodiment, the pharmaceutical composition for use according to the invention comprises a vanadium compound wherein the ligands of the vanadium(IV) are antioxidants other than maltol.

In one embodiment, the pharmaceutical composition for use according to the invention comprises a vanadium compound wherein said vanadium compound is combined with antioxidants.

In an alternative embodiment, the pharmaceutical composition comprising a physiologically acceptable organic and/or inorganic vanadium compound or complex for use according to the invention comprises in addition to the physiologically acceptable organic and/or inorganic vanadium compound or complex a further active pharmaceutical ingredient.

In one embodiment, the pharmaceutical composition comprising BMOV or BEOV or a combination of BMOV and BEOV for use according to the invention comprises in addition to said BMOV or BEOV or a combination of BMOV and BEOV a further active pharmaceutical ingredient. Typically, such a further active pharmaceutical ingredient comprised by the pharmaceutical composition of the invention is a compound for lowering the glucose level in blood when administered to a subject such as a human subject. Examples of such blood glucose lowering compounds include insulin, either for oral administration, or for parenteral administration including intradermal and intramuscular injection.

Examples of blood glucose lowering agents which can be combined with a physiologically acceptable organic and/or inorganic vanadium compound or complex such as BMOV, BEOV or the combination of BMOV and BEOV in the pharmaceutical composition comprising a physiologically acceptable organic and/or inorganic vanadium compound or complex for use according to the invention, are manifold. Such blood lowering agents, as said including insulins, are compounds suitable for parenteral administration and/or suitable for oral administration, or for example suitable for inhalation when provided as a mist or the like. Without wanted to be limited to the following examples, the pharmaceutical composition comprising a physiologically acceptable organic and/or inorganic vanadium compound or complex according to the invention may further comprise any one or more of the list of the following compounds for lowering blood glucose level in a (human) subject: a biguanide such as metformin, metformin liquid and metformin extended release formulation; a sulfonylureas such as glimepiride, glyburide, glipizide, micronized glyburide, meglitinides, repaglinide; a D-Phenylalanine derivative such as nateglinide; a thiazolidinedione such as pioglitazone, pioglitazone; a DPP-4 Inhibitor such as sitagliptin, saxagliptin, sinagliptin; an alpha-glucosidase Inhibitor, such as acarbose, miglitol; a bile Acid Sequestrant such as colesevelam; combination preparations, such as provided as a pill, including pioglitazone & metformin, glyburide & metformin, glipizide & metformin, sitagliptin & metformin, saxagliptin & metformin, repaglinide & metformin, pioglitazone & glimepiride, metformin-alogliptin, metformin-canagliflozin, metformin-dapagliflozin, metformin-empagliflozin, metformin-glipizide, metformin-glyburide, metformin-linagliptin, metformin-pioglitazone, metformin-repaglinide, metformin-rosiglitazone, metformin-saxagliptin, metformin-sitagliptin; insulin for injection such as short-acting insulin and rapid-acting insulin such as insulin aspart, insulin glulisine, insulin lispro, or intermediate-acting insulin such as insulin isophane, long-acting insulins such as insulin degludec, insulin detemir, insulin glargine, combination insulins such as NovoLog Mix 70/30 (insulin aspart protamine-insulin aspart), Humalog Mix 75/25 (insulin lispro protamine-insulin lispro), Humalog Mix 50/50 (insulin lispro protamine-insulin lispro), Humulin 70/30 (human insulin NPH-human insulin regular), Novolin 70/30 (human insulin NPH-human insulin regular), Ryzodeg (insulin degludec-insulin aspart), an amylinomimetic drug such as pramlintide; a dopamine agonist such as bromocriptine; any one or more of alogliptin, alogliptin-metformin, alogliptin-pioglitazone, linagliptin, linagliptin-empagliflozin, linagliptin-metformin, saxagliptin, saxagliptin-metformin, sitagliptin, sitagliptin-metformin, sitagliptin and simvastatin; a glucagon-like peptide (incretin mimetic) such as albiglutide, dulaglutide, exenatide, exenatide extended-release, liraglutideMeglitinides; nateglinide, repaglinide and/or repaglinide-metformin; a odium glucose transporter (SGLT) 2 inhibitor such as dapagliflozin, dapagliflozin-metformin, canagliflozin, canagliflozin-metformin, empagliflozin, empagliflozin-linagliptin, empagliflozin-metformin; a sulfonylurea such as glimepiride, glimepiride-pioglitazone, glimeperide-rosiglitazone, gliclazide, glipizide, glipizide-metformin, glyburide, glyburide-metformin, chlorpropamide, tolazamide, tolbutamide; a thiazolidinedione such as rosiglitazone, rosiglitazone-glimepiride, rosiglitizone-metformin, pioglitazone, pioglitazone-alogliptin, pioglitazone-glimepiride, pioglitazone-metformin, to name a few.

Of course, a dosage regimen according to the invention can also consist of providing the pharmaceutical composition comprising a physiologically acceptable organic and/or inorganic vanadium compound or complex such as BMOV for use according to the invention and separately providing any one or more of the above listed blood glucose lowering agents other than a physiologically acceptable organic and/or inorganic vanadium compound or complex such as BMOV. In one embodiment, the dosage regimen encompasses the provision of the pharmaceutical composition comprising a physiologically acceptable organic and/or inorganic vanadium compound or complex such as BMOV as the sole pharmaceutical ingredient for use according to the invention and the separate provision of any one or more of the above listed blood glucose lowering agents other than a physiologically acceptable organic and/or inorganic vanadium compound or complex such as BMOV.

In one embodiment, the pharmaceutical composition of the invention comprises a physiologically acceptable organic and/or inorganic vanadium compound or complex such as BMOV and further comprises a second active pharmaceutical ingredient for use according to the invention. Preferred is a pharmaceutical composition of the invention comprises a physiologically acceptable organic and/or inorganic vanadium compound or complex such as BMOV and further comprising a second active pharmaceutical ingredient for use according to the invention, wherein the second active pharmaceutical ingredient is an insulin.

In one embodiment, the pharmaceutical composition of the invention comprises a physiologically acceptable organic and/or inorganic vanadium compound or complex such as BMOV and further comprises a second active pharmaceutical ingredient for use according to the invention, wherein an insulin is administered to the patient separate from the pharmaceutical composition. Typically, the insulin is then administered at the same time point(s) at which the pharmaceutical composition for use according to the invention is administered, before and during stress-induced hyperglycemia occurs, or before and during and after stress-induced hyperglycemia occurs, or before stress-induced hyperglycemia occurs, and/or the insulin is administered at separate time points, according to the invention. Equally preferred is the similar administration regime of a second active pharmaceutical ingredient other than insulin, wherein said second active pharmaceutical ingredient has blood glucose level lowering activity once administered to a patient, according to the invention.

In one embodiment, the pharmaceutical composition of the invention comprises a physiologically acceptable organic and/or inorganic vanadium compound or complex such as BMOV for use according to the invention, or the physiologically acceptable organic and/or inorganic vanadium compound or complex such as BMOV is the sole active pharmaceutical ingredient, wherein during the occurrence of stress in the subject, i.e. the patient, also insulin is administered to the subject. Without wishing to be bound by theory, the blood glucose lowering capacity of for example BMOV is enhanced when insulin is present in the circulation of the patient, i.e. a level of insulin that is above the threshold level for enhancing the effect of e.g. BMOV. Therefore, if the patient to whom the pharmaceutical composition for use according to the invention is administered, has an insulin level in the circulation that is below such threshold for enhancing the activity of e.g. BMOV, the skilled person will appreciate that the patient benefits from the administration of an amount of insulin resulting in near normal level of insulin in the circulation of the patient. According to the invention, the required amount of insulin to raise the insulin level in the circulation of the patient to a level at which for example BMOV activity is enhanced, is lower than the required amount of insulin to treat hyperglycemia in a patient suffering from e.g. acute stress, when such a patient is treated with insulin therapy only.

The subject, such as an animal such as a mammal, preferably a human subject such as a diabetic human subject or a non-diabetic human subject, to whom the pharmaceutical composition comprising a physiologically acceptable organic and/or inorganic vanadium compound or complex such as BMOV, BEOV or the combination of BMOV and BEOV for use according to the invention is administered before said subject is subjected to stress is a healthy subject, according to the invention. Alternatively, the subject is a diabetes patient, i.e. either suffering from diabetes mellitus type I, or suffering from diabetes mellitus type II, according to the invention. Further, the human subject to whom a certain stress will be elicited and to whom the pharmaceutical composition comprising a physiologically acceptable organic and/or inorganic vanadium compound or complex such as BMOV for use according to the invention is administered before the stress is elicited, may be a patient suffering from any health problem or disease, either resulting in derailed glycemic control in the subjects body, or not, said health problem or disease for example being cardiovascular disease, atherosclerosis, malfunctioning of any organ such as the kidney, liver, lungs, brain, skin, intestines, etc.

In one embodiment, the pharmaceutical composition for use according to the invention is administered to the patient at least once at a time point between 96 h and 0 h before the patient is subjected to the stress, preferably at a time point between 72 h and 1 h, more preferably at a time point between 48 h and 2 h, most preferably at a time point between 24 h and 3 h before the patient is subjected to the stress. For example, BMOV is active with regard to the prevention or reduction of stress-induced metabolic derangement in a patient subjected to stress, in the subject's body to whom a dose of BMOV is administered for, for example, at least 72 days, such as for example four to five days, depending on the dose that is administered to said subject. In one embodiment, the pharmaceutical composition for use according to the invention is administered to the patient at least once at a time point between 30 h and 0 h before the patient is subjected to the stress, preferably at a time point between 24 h and 2 h, more preferably at a time point between 24 h and 3 h before the patient is subjected to the stress. Preferably, the pharmaceutical composition for use according to the invention is administered to the patient at least twice, at a first time point between 30 h and 10 h before the patient is subjected to the stress, preferably at a time point between 24 h and 12 h, more preferably at a time point between 24 h and 16 h before the patient is subjected to the stress, and at a second time point between 8 h and 0 h before the patient is subjected to the stress, preferably at a time point between 5 h and 2 h, more preferably at about 3 h before the patient is subjected to the stress. Preferably, the pharmaceutical composition for use according to the invention is administered to the patient at least twice, at a first time point between 96 h and 10 h before the patient is subjected to the stress, preferably at a time point between 72 h and 12 h, more preferably at a time point between 48 h and 14 h, most preferably at a time point between 24 h and 16 h before the patient is subjected to the stress, and at a second time point between 8 h and 0 h before the patient is subjected to the stress, preferably at a time point between 5 h and 2 h, more preferably at about 3 h before the patient is subjected to the stress.

As is evident from the exemplifying experimental in vivo results in mammals, e.g. rats (see Examples section), subjects are prevented against the occurrence of hyperglycemia when the subjects are pretreated with a pharmaceutical composition comprising BMOV, e.g. a pharmaceutical composition comprising BMOV wherein the BMOV is the sole active pharmaceutical ingredient, before being exposed to stress, according to the invention. As exposure of a subject to stress is of course not in all occasions anticipated and/or foreseeable, chronic treatment with a pharmaceutical composition comprising BMOV is a measure for the prevention or amelioration of the induction of hyperglycemia as a result of exposure to stress. That is to say, a subject is administered a dose of a pharmaceutical composition comprising BMOV before the subject is subjected to stress, wherein the dosage regimen comprises administering the BMOV once daily, or twice daily, for one day before the anticipated stress is induced, or for a prolonged period of time as a preventive measure in case stress may be induced to the subject, or even for a life time if exposure to stress is foreseeable or if a subject has an (increased) risk for being subjected to stress in the (near) future, e.g. within a number of days, weeks, months. For example workers, such as workers who are being send to a life threatening situation, for example soldiers, firemen, emergency aid, policemen, or sportsmen at relatively high risk of being subjected to stress such as trauma-related stress, are subjected to a dosage regimen comprising the chronic administration of a pharmaceutical composition comprising BMOV for a prolonged period of time in which the risk for receiving e.g. a trauma is increased. For example, human subjects at risk for receiving a trauma are administered a pharmaceutical composition comprising BMOV twice daily, according to the invention. Preferably, the pharmaceutical composition of the invention is administered to a subject in need thereof by injection, according to the invention, although oral administration is also suitable. Of course, monitoring of glucose level in the blood is part of such dosage regimen, in order to monitor whether normoglycemia is maintained and no hypoglycemia occurs.

In one embodiment, the pharmaceutical composition for use according to the invention is used in the prevention or reducing hyperglycemia in a subject, such as a human subject, such as a healthy subject, who is subjected to a stress, wherein said stress is a trauma, such as a trauma caused by surgery, a trauma such as a medical trauma caused by a medical procedure, a trauma caused by iatrogenic injury, and/or wherein the stress to which the patient is subjected is caused by any one or more of an infection, administration of one or more medicine(s), either long term or short term acute mental stress due to a life threatening situation, such as a potentially life threatening situation and an accident such as a car accident or a collapsed building capturing a subject, according to the invention. The origin and cause of the stress which results in hyperglycemia in a subject exposed to such stress, is irrelevant for the mode of action of the pharmaceutical composition comprising a physiologically acceptable organic and/or inorganic vanadium compound or complex such as BMOV of the invention when the physiologically acceptable organic and/or inorganic vanadium compound or complex such as BMOV is administered to a subject before the subject experiences the stress conditions. Thus, the stress may relate to trauma due to any cause (e.g. surgery, accident, sports match, etc.), or may relate to infection, sepsis, inflammation, organ failure, malfunctioning of an organ or of tissue, burns such as sun burn, cancer, undercooling of the subject, overheating of the subject, dehydration of the subject, shock, heart failure, blood loss, low blood pressure, high blood pressure, adrenaline rise in the blood, suffocation, reperfusion (such as after a heart attack or after surgery), ischemia, broken and fractured bones, etc.

In one embodiment, the pharmaceutical composition for use according to the invention, wherein the pharmaceutical composition comprising a physiologically acceptable organic and/or inorganic vanadium compound or complex such as BMOV is administered orally to the patient, or administered intravenously to the patient, or administered intraperitoneally, or administered intramuscularly, or administered intradermally, preferably the pharmaceutical composition comprising is administered intravenously to the patient or intradermally or intraperitoneally.

The pharmaceutical composition of the invention is suitable for many of the commonly applied routes of administration of pharmaceutical compositions to a subject, such as a human subject. The pharmaceutical composition of the invention is applicable for administering to a subject in need thereof via noninvasive routes such as orally, nasally, vaginally, rectally, pulmonarily, in the eye (eye drops), sublingually, in the ear, transdermally (T.D.), and is applicable for administering to a subject in need thereof via invasive routes such as intradermally (I.D.), subcutaneously (S.C.), intramuscularly (I.M.), intravenously (I.V.), intra-arterially (I.A.), intra-thecally (I.T.), intraperitoneally (I.P.), intra-articularly (synovial fluids), and intra-ventricularly.

It is one of the many benefits of the current invention that a subject who will foreseeably be subjected to stress in the near feature, e.g. within a day or a couple of days, benefits from pretreatment with the pharmaceutical composition comprising a physiologically acceptable organic and/or inorganic vanadium compound or complex such as BMOV for use according to the invention, before the subject is subjected to the stress. Most conveniently, such pretreatment comprises a dosage regimen consisting of a formulation comprising a physiologically acceptable organic and/or inorganic vanadium compound or complex such as BMOV or BEOV or the combination of BMOV and BEOV which can be administered orally. Therefore, the pharmaceutical composition comprising a physiologically acceptable organic and/or inorganic vanadium compound or complex such as BMOV of the invention is provided as a formulation for oral use, such as a tablet, a powder, a pill. Preferably, a dosage regimen of the invention comprises administering orally a single pill or tablet or the like comprising the pharmaceutical composition for use according to the invention. Of course, a dosage regimen may also comprise administering at least two tablets, etc. in order to administer to a subject a single dose of the pharmaceutical composition for use according to the invention, although a single tablet, pill, etc. comprising a full single dose of a physiologically acceptable organic and/or inorganic vanadium compound or complex such as BMOV is preferred. It is part of the invention that before the subjection of a subject to stress, the dose of a physiologically acceptable organic and/or inorganic vanadium compound or complex such as BMOV is administered in a time frame of between 0 h and 96 h before the onset of a stress, wherein the pharmaceutical composition is administered as a plurality of dose units in order to reduce the risk of side effects inflicted by the pharmaceutical composition, such as gastro-intestinal side effects, according to the invention. Of course, the skilled person will appreciate that the optimal dosage and number of administered doses are selected in relation to the desired effective plasma levels of the physiologically acceptable organic and/or inorganic vanadium compound or complex such as BMOV at the time the stress is elicited to the subject and thereafter.

In one embodiment, the pharmaceutical composition for use according to the invention is an aqueous solution, preferably an aqueous solution comprising between about 0.001 mg and 10 mg of a physiologically acceptable organic and/or inorganic vanadium compound or complex per ml of the pharmaceutical composition, preferably between about 0.5 mg/ml and 5 mg/ml, more preferably between about 2 mg of a physiologically acceptable organic and/or inorganic vanadium compound or complex per ml. Such aqueous solution is beneficially applied in a dosage regimen comprising orally administering or intravenously administering or dermally administering by injection or intramuscularly administering by e.g. injection a single dose of the pharmaceutical composition for use according to the invention to a subject in need thereof, for the pretreatment of the subject before stress is elicited to the subject.

In one embodiment, the pharmaceutical composition for use according to the invention is an aqueous solution, preferably an aqueous solution comprising between about 0.5 mg BMOV per ml and 5 mg BMOV per ml of the pharmaceutical composition, preferably about 2 mg BMOV per ml. In an alternative embodiment, the physiologically acceptable organic and/or inorganic vanadium compound or complex is BEOV or a combination of BMOV and BEOV.

In one embodiment, the pharmaceutical composition for use according to the invention, comprises at least one pharmaceutically acceptable excipient, said pharmaceutically acceptable excipient comprising a phosphate buffered saline, said phosphate buffered saline comprising 1.86 g/l NaH₂PO₄. H₂O, 9.50 g/l Na₂HPO₄. 2 H₂O and 4.40 g/l NaCl, preferably said pharmaceutically acceptable excipient consists of phosphate buffered saline consisting of about 1.86 g/l NaH₂PO₄. H₂O, about 9.50 g/l Na₂HPO₄. 2 H₂O and about 4.40 g/l NaCl. Such phosphate buffered saline composition is a particular suitable pharmaceutically acceptable excipient for the administration of the pharmaceutical composition comprising BMOV for use according to the invention, in particular when the composition is for intravenous administration, intramuscular administration, intradermal administration, although oral administration is also possible. Typically, the pH of a phosphate buffered saline for use as the pharmaceutically acceptable excipient in the pharmaceutical composition for use according to the invention, is near physiological, such as a pH of between 6 and 8, preferably between 6.5 and 7.5. Typically the pH of such a pharmaceutically acceptable excipient is between about 7.2 and 7.4.

Of course, other aqueous solutions may serve as a pharmaceutically acceptable excipient in the pharmaceutical composition comprising a physiologically acceptable organic and/or inorganic vanadium compound or complex such as BMOV for use according to the invention, as well. For example, a pharmaceutically acceptable excipient suitable for application in intravenous administration and/or intradermal administration and/or inhalation as a mist in the lungs and/or intramuscular administration and/or oral administration, is equally suitable for application in the pharmaceutical composition comprising a physiologically acceptable organic and/or inorganic vanadium compound or complex such as BMOV for use according to the invention, according to the invention. Such pharmaceutically acceptable excipients are well known in the art.

In one embodiment, the pharmaceutical composition for use according to the invention is administered orally to the patient, wherein the pharmaceutical composition comprising a physiologically acceptable organic and/or inorganic vanadium compound or complex such as BMOV is provided as a capsule, a tablet, a pill or a water-miscible powder for oral administration. Of course, any other formulation of the pharmaceutical composition comprising a physiologically acceptable organic and/or inorganic vanadium compound or complex such as BMOV for use according to the invention, suitable for oral administration, is equally suitable for the purpose of preventing or ameliorating metabolic derangement, in particular hyperglycemia, in a patient who is to be subjected to a stress such as trauma related to surgery, according to the invention.

In one embodiment, the pharmaceutical composition comprising a physiologically acceptable organic and/or inorganic vanadium compound or complex such BMOV or BEOV for use according to the invention, is used as a single dose of the pharmaceutical composition administered to the patient, or is used in a dosage regimen being administration of at least two doses of the pharmaceutical composition comprising a physiologically acceptable organic and/or inorganic vanadium compound or complex such as BMOV or BEOV to the patient per between 12 h and 96 h, preferably two doses per between 16 h and 72 h. In one embodiment, the pharmaceutical composition comprising a physiologically acceptable organic and/or inorganic vanadium compound or complex such BMOV or BEOV for use according to the invention, is used as a single dose of the pharmaceutical composition administered to the patient, or is used in a dosage regimen being administration of at least two doses of the pharmaceutical composition comprising a physiologically acceptable organic and/or inorganic vanadium compound or complex such as BMOV or BEOV to the patient per between 12 h and 24 h, preferably two doses per between 16 h and 24 h.

In one embodiment, the pharmaceutical composition comprising a physiologically acceptable organic and/or inorganic vanadium compound or complex such as BMOV for use according to the invention, is administered at a dose of pharmaceutical composition comprising the physiologically acceptable organic and/or inorganic vanadium compound or complex to the patient comprising between 7,5 mg and 30 mg of the physiologically acceptable organic and/or inorganic vanadium compound or complex per kg body weight of the patient, preferably between 12 mg and 20 mg of the physiologically acceptable organic and/or inorganic vanadium compound or complex per kg body weight of the patient, more preferably about 15 mg of the physiologically acceptable organic and/or inorganic vanadium compound or complex per kg body weight of the patient, wherein the physiologically acceptable organic and/or inorganic vanadium compound or complex is preferably BMOV.

In one embodiment, the pharmaceutical composition comprising a physiologically acceptable organic and/or inorganic vanadium compound or complex such as BMOV for use according to the invention, is administered to the subject such as a human at a dose of pharmaceutical composition comprising between 0.01 mg and 30 mg of the physiologically acceptable organic and/or inorganic vanadium compound or complex per kg body weight of the patient, preferably between 0.1 mg and 15 mg of the physiologically acceptable organic and/or inorganic vanadium compound or complex per kg body weight of the patient, more preferably between 0.15 mg/kg and 10 mg/kg, most preferably between 0.5 mg and 2.5 mg of the physiologically acceptable organic and/or inorganic vanadium compound or complex per kg body weight of the patient, wherein the physiologically acceptable organic and/or inorganic vanadium compound or complex is preferably BMOV. It has to be acknowledged that these dose levels in mg are based on the molecular weight of BMOV and that for other vanadium containing compounds or complexes absolute dose levels should reflect an equimolar amount of vanadium. In addition, these dose levels reflect intravenous administration. Irrespective of the specific vanadium containing compound and/or route of administration, the dose level and administration should result preferably in general plasma exposure levels between 25 ng/ml and 2500 ng/ml elemental vanadium or more preferably between 100 ng/ml and 1000 ng/ml, notwithstanding that peak plasma levels may be exceeding these limits. Typically, such plasma exposure levels are reached in subjects, preferably human subjects there were administered a physiologically acceptable vanadium compound or vanadium complex such as BMOV, BEOV, orally, or subcutaneously, or intraperitoneally, or intravenously. Typically, such plasma exposure levels of between 25 ng/ml and 2500 ng/ml such as between 100 ng/ml and 1000 ng/ml are for example obtained by i.v. administration of BMOV to a human subject. Alternatively, such levels are reached upon oral administration of BEOV or vanadium sulphate to human diabetic subjects. In one embodiment, the pharmaceutical composition comprising a physiologically acceptable organic and/or inorganic vanadium compound or complex such as BMOV for use according to the invention, is administered to the subject such as a human at a dose of pharmaceutical composition comprising between 0.5 mg and 4 mg of the physiologically acceptable organic and/or inorganic vanadium compound or complex per kg body weight of the patient.

In one embodiment, the pharmaceutical composition comprising a physiologically acceptable organic and/or inorganic vanadium compound or complex such as BMOV for use according to the invention, is administered to the subject such as a human at a dose of pharmaceutical composition comprising between 0.001 mg and 30 mg of the physiologically acceptable organic and/or inorganic vanadium compound or complex per kg body weight of the patient, such that the plasma exposure level of the vanadium compound or complex such as BMOV or BEOV in the subject, e.g. a human subject is between 25 ng/ml and 2500 ng/ml elemental vanadium or more preferably between 100 ng/ml and 1000 ng/ml. Such listed plasma exposure levels are notwithstanding that peak plasma levels may be exceeding these limits.

In one embodiment, the pharmaceutical composition comprising a physiologically acceptable organic and/or inorganic vanadium compound or complex such as BMOV for use according to the invention, is administered at a dose of pharmaceutical composition comprising the physiologically acceptable organic and/or inorganic vanadium compound or complex to the patient comprising between 1 mg and 30 mg of a physiologically acceptable organic and/or inorganic vanadium compound or complex per kg body weight of the patient, preferably between 4 mg and 20 mg of a physiologically acceptable organic and/or inorganic vanadium compound or complex per kg body weight of the patient, more preferably between about 6 mg and 12 mg of a physiologically acceptable organic and/or inorganic vanadium compound or complex per kg body weight of the patient, wherein the physiologically acceptable organic and/or inorganic vanadium compound or complex is preferably BMOV. As said before, it has to be acknowledged that these dose levels in mg according to the invention are based on the molecular weight of BMOV and that for other vanadium containing compounds or complexes absolute dose levels should reflect an equimolar amount of vanadium. In addition, these dose levels reflect intravenous administration of the pharmaceutical composition for use according to the invention. Irrespective of the specific vanadium compound or vanadium complex and/or irrespective of the chosen route of administration, the particular dose level and particular route of administration should result preferably in general plasma exposure levels between 25 ng/ml and 2500 ng/ml elemental vanadium or more, more preferably between 100 ng/ml and 1000 ng/ml, notwithstanding that peak plasma levels may be exceeding these limits.

Highly preferred is administering the pharmaceutical composition comprising a physiologically acceptable organic and/or inorganic vanadium compound or complex such as BMOV for use according to the invention to a patient in the time period before the patient is subjected to a stress, such as the stress relating to trauma during surgery. In one embodiment, the pharmaceutical composition comprising a physiologically acceptable organic and/or inorganic vanadium compound or complex such as BMOV for use according to the invention is administered to the patient in the time period in which the patient is subjected to the stress. Alternatively, in one embodiment, the pharmaceutical composition comprising a physiologically acceptable organic and/or inorganic vanadium compound or complex such as BMOV for use according to the invention is administered to the patient in the time period before the patient is subjected to a stress and subsequently also in the time period in which the patient is subjected to the stress, such as the stress relating to undergoing surgery accompanied by inducing a trauma in the patient. Of course, if a patient who is subjected to stress is in need thereof, also pharmaceutical composition comprising physiologically acceptable organic and/or inorganic vanadium compound or complex such as BMOV for use according to the invention is administered to said patient after the patient is relieved from the stress that is for example elicited by surgery, though is for example recovering from said stress (e.g. wound healing after an accident, after surgery). The effect of the pre-treatment of the patient who will be subjected to a stress or who is subjected to a stress or who is recovering from being subjected to a stress, with the pharmaceutical composition comprising a physiologically acceptable organic and/or inorganic vanadium compound or complex such as BMOV for use according to the invention is the same: maintenance of normoglycemia or reducing the extent of a rise in glucose level in the blood of the patient during the period of stress and/or thereafter. As said, said pre-treatment with the pharmaceutical composition comprising a physiologically acceptable organic and/or inorganic vanadium compound or complex such as BMOV can be supplemented with continued treatment with the pharmaceutical composition comprising a physiologically acceptable organic and/or inorganic vanadium compound or complex such as BMOV for use according to the invention during the period of exposure of the patient to stress and/or after said period of stress, according to the invention.

In one embodiment, the pharmaceutical composition comprising a physiologically acceptable organic and/or inorganic vanadium compound or complex such as BMOV for use according to the invention is administered to the patient in the time period before the patient is subjected to a stress and optionally subsequently also in the time period in which the patient is subjected to the stress and optionally subsequently also in the time period in which the patient that was subjected to stress and in which post-treatment time period the patient is recovering from the stress and its consequences, e.g. the insult, trauma, acute insult, tissue regeneration and/or wound healing after e.g. trauma such as caused by infection, surgery, an accident, etc.

In one embodiment, the pharmaceutical composition comprising a physiologically acceptable organic and/or inorganic vanadium compound or complex such as BMOV for use according to the invention is administered as a first single dose to the patient at a time point of between 96 h and 10 h, preferably between 72 h and 12 h, more preferably between 48 h and 14 h, most preferably between 24 h and 16 h before the patient is subjected to the stress and a second single dose of the pharmaceutical composition is administered to the patient at a time point of between 8 h and 0 h, preferably between 5 h and 2 h before the patient is subjected to the stress, preferably said first single dose is administered between 24 h and 16 h and said second single dose is administered about 3 h before the patient is subjected to the stress.

In one embodiment, the pharmaceutical composition comprising a physiologically acceptable organic and/or inorganic vanadium compound or complex such as BMOV for use according to the invention is administered as a first single dose to the patient at a time point of between 96 h and 10 h, preferably between 72 h and 12 h, more preferably between 48 h and 14 h, most preferably between 24 h and 16 h before the patient is subjected to the stress and a second single dose of the pharmaceutical composition is administered to the patient at a time point of between 8 h and 0 h, preferably between 5 h and 3 h before the patient is subjected to the stress, preferably said first single dose is administered between 24 h and 16 h and said second single dose is administered about 3 h before the patient is subjected to the stress. In one embodiment, the pharmaceutical composition comprising a physiologically acceptable organic and/or inorganic vanadium compound or complex such as BMOV for use according to the invention is administered as a first single dose to the patient at a time point of between 24 h and 12 h, preferably between 24 h and 16 h before the patient is subjected to the stress, and is administered as a second single dose to the patient at a time point of between 8 h and 0 h, preferably between 5 h and 3 h before the patient is subjected to the stress, preferably said first single dose is administered between 24 h and 16 h and said second single dose is administered about 3 h before the patient is subjected to the stress.

In one embodiment, the pharmaceutical composition comprising a physiologically acceptable organic and/or inorganic vanadium compound or complex such as BMOV for use according to the invention is administered as a first single dose to the patient at a time point of between 96 h and 0 h, preferably between 72 h and 1 h, more preferably between 48 h and 2h, most preferably between 24 h and 3 h before the patient is subjected to the stress and a second single dose of the pharmaceutical composition is administered to the patient during the period in which the patient is subjected to the stress. In one embodiment, the pharmaceutical composition comprising a physiologically acceptable organic and/or inorganic vanadium compound or complex such as BMOV for use according to the invention is administered as a first single dose to the patient at a time point of between 24 h and 0 h, preferably between 20 h and 3 h before the patient is subjected to the stress, and is administered as a second single dose to the patient during the period in which the patient is subjected to the stress.

In one embodiment, the pharmaceutical composition comprising a physiologically acceptable organic and/or inorganic vanadium compound or complex such as BMOV for use according to the invention is administered as a first single dose to the patient at a time point of between 96 h and 10 h, preferably between 72 h and 12 h, more preferably between 48 h and 14 h, most preferably between 24 h and 16 h before the patient is subjected to the stress and a second single dose of the pharmaceutical composition is administered to the patient at a time point of between 8 h and 0 h, preferably between 5 h and 3 h before the patient is subjected to the stress and a third single dose of the pharmaceutical composition is administered to the patient during the period in which the patient is subjected to the stress. In one embodiment, the pharmaceutical composition comprising a physiologically acceptable organic and/or inorganic vanadium compound or complex such as BMOV for use according to the invention is administered as a first single dose to the patient at a time point of between 24 h and 12 h, preferably between 24 h and 16 h before the patient is subjected to the stress, and is administered as a second single dose to the patient at a time point of between 8 h and 0 h, preferably between 5 h and 3 h before the patient is subjected to the stress, and is administered as a third single dose to the patient during the period in which the patient is subjected to the stress.

In one embodiment, the pharmaceutical composition comprising a physiologically acceptable organic and/or inorganic vanadium compound or complex such as BMOV for use according to the invention is provided as an aqueous solution or as a solid dosage form such as a powder, a tablet, a pill and a capsule.

In one embodiment, the pharmaceutical composition comprising a physiologically acceptable organic and/or inorganic vanadium compound or complex such as BMOV for use according to the invention is provided such that a dose of the pharmaceutical composition comprises between about 0.1 mg and 40 mg of the physiologically acceptable organic and/or inorganic vanadium compound or complex such as BMOV per kg body weight of the patient, preferably between about 0.5 mg and 1 mg of the physiologically acceptable organic and/or inorganic vanadium compound or complex such as BMOV per kg body weight of the patient.

In one embodiment, the pharmaceutical composition comprising a physiologically acceptable organic and/or inorganic vanadium compound or complex such as BMOV for use according to the invention is provided such that a dose of the pharmaceutical composition comprises between about 0.01 mg and 30 mg of the physiologically acceptable organic and/or inorganic vanadium compound or complex such as BMOV per kg body weight of the patient, preferably about 5 mg, more preferably between about 0.5 mg and 2.5 mg of the physiologically acceptable organic and/or inorganic vanadium compound or complex such as BMOV per kg body weight of the patient.

In one embodiment, the pharmaceutical composition comprising a physiologically acceptable organic and/or inorganic vanadium compound or complex such as BMOV for use according to the invention is provided such that a dose of the pharmaceutical composition comprises between about 0.001 mg and 30 mg of the physiologically acceptable organic and/or inorganic vanadium compound or complex such as BMOV per kg body weight of the patient, preferably about 5 mg, more preferably between about 0.5 mg and 2.5 mg of the physiologically acceptable organic and/or inorganic vanadium compound or complex such as BMOV per kg body weight of the patient, wherein the pharmaceutical composition is provided as an aqueous solution or as a solid dosage form such as a powder, a tablet, a pill, and a capsule. Again, as outlined before, irrespective of the specific vanadium containing compound or complex and/or irrespective of the route of administration, e.g. oral, i.v., i.p., s.c, the dose level and administration should result preferably in general plasma exposure levels between 25 ng/ml and 2500 ng/ml elemental vanadium or more preferably between 100 ng/ml and 1000 ng/ml, notwithstanding that peak plasma levels may be exceeding these limits. Typically, such plasma exposure levels are reached in subjects, preferably human subjects there were administered a physiologically acceptable vanadium compound or vanadium complex such as BMOV, BEOV, orally, or subcutaneously, or intraperitoneally, or intravenously.

In one embodiment, the pharmaceutical composition comprising a physiologically acceptable organic and/or inorganic vanadium compound or complex such as BMOV for use according to the invention is provided as an aqueous solution or as a solid dosage form such as a powder, a tablet, a pill, and a capsule, and wherein a dose of the pharmaceutical composition comprises between about 0.01 mg and 30 mg of the physiologically acceptable organic and/or inorganic vanadium compound or complex per kg body weight of the patient, wherein administering a dose of the pharmaceutical composition to said patient provides for a plasma exposure level of between 25 ng/ml and 2500 ng/ml elemental vanadium, or preferably between 100 ng/ml and 1000 ng/ml in said patient.

In one embodiment, the pharmaceutical composition comprising a physiologically acceptable organic and/or inorganic vanadium compound or complex such as BMOV for use according to the invention is administered to a patient, wherein the stress to which said patient is subjected to is a trauma, such as a trauma caused by surgery, a medical trauma caused by a medical procedure, a trauma caused by iatrogenic injury, and/or wherein the stress to which the patient is subjected is caused by any one or more of an infection, administration of one or more medicine(s), either long term or short term acute mental stress due to a life threatening situation, such as a potentially life threatening situation and an accident. In fact, use of the pharmaceutical composition comprising a physiologically acceptable organic and/or inorganic vanadium compound or complex such as BMOV of the invention for the prevention or amelioration of stress-induced hyperglycemia is not dependent on the origin of the stress, the stress being any type of trauma to any extent, the trauma caused by any event such as surgery-related trauma or a wound caused for example by sports, an accident, etc.

In one embodiment, the pharmaceutical composition comprising a physiologically acceptable organic and/or inorganic vanadium compound or complex such as BMOV for use according to the invention is used in a dosage regimen such that the increase of glucose content in the blood of the patient during the period in which the patient is subjected to the stress is less than about 100%, preferably less than about 65%, more preferably less than about 30%, most preferably less than about 6%, compared to the glucose content in the blood of the patient at a time point 3 h to 0 minute before the patient is subjected to the stress, preferably compared to the glucose content in the blood of the patient at the time point at which the patient starts to be subjected to the stress. An important benefit of the present invention is the provision of a pretreatment modality useful for the prevention of hyperglycemia in subjects subjected to stress, i.e. the pharmaceutical composition comprising a physiologically acceptable organic and/or inorganic vanadium compound or complex such as BMOV for use according to the invention, is the prevention of a rise in the glucose level in blood that would otherwise occur upon the stress elicited to the subject, and would then result in health risks, organ damage, etc. As outlined before maintenance of normoglycemia, i.e. the presence of a normal concentration of glucose in the blood, not giving rise to an increased risk for health problem or for initiating disease or damage to tissue, veins, arteries, etc., is of utmost importance and highly beneficial to the health status of a subject exposed to stress.

In one embodiment, the pharmaceutical composition comprising a physiologically acceptable organic and/or inorganic vanadium compound or complex such as BMOV for use according to the invention is used in a dosage regimen such that the increase of glucose content in the blood of the patient during the first one to eight hours, preferably during the first about three hours of the period in which the patient is subjected to the stress is less than about 100%, preferably less than about 65%, more preferably less than about 30%, most preferably less than about 6%, compared to the glucose content in the blood of the patient at a time point 3 h to 0 minute before the patient is subjected to the stress, preferably compared to the glucose content in the blood of the patient at the time point at which the patient starts to be subjected to the stress.

In one embodiment, the pharmaceutical composition comprising a physiologically acceptable organic and/or inorganic vanadium compound or complex such as BMOV for use according to the invention is used in a dosage regimen such that the increase of glucose content in the blood of the patient during the first one to eight hours, preferably during the first about three hours of the period in which the patient is subjected to the stress is between about -25% and about 100%, preferably between about 0% and about 75%, more preferably between about 5% and about 65%, most preferably about 30%, compared to the glucose content in the blood of the patient at a time point 3 h to 0 minute before the patient is subjected to the stress, preferably compared to the glucose content in the blood of the patient at the time point at which the patient starts to be subjected to the stress.

In one embodiment, the pharmaceutical composition comprising a physiologically acceptable organic and/or inorganic vanadium compound or complex such as BMOV for use according to the invention is used in a pretreatment regimen before the patient is exposed to a stress or in a treatment regimen including both pretreatment of the patient before the patient is subjected to the stress and treatment of the patient during the time period wherein the patient is exposed to the stress. In one embodiment, the pharmaceutical composition comprising a physiologically acceptable organic and/or inorganic vanadium compound or complex such as BMOV for use according to the invention is used in said (pre)treatment regimen, wherein the stress that is subjected to the patient is stress caused by trauma occurring during any one or more of surgery such as cardiac surgery, amputation surgery, tumor resection surgery, brain surgery, transplantation surgery, surgery related to restoring hemostasis, surgery related to treating bone fracture, surgery related to organ or bone restoration such as hip transplant surgery, blood vessel surgery, bypass surgery, closing of wounds, treatment of injuries to the skin, bones, organs, tissue, arteries, veins, a medical procedure, iatrogenic injury, an infection, administration of one or more medicine(s), acute mental stress, an accident.

In one embodiment, the pharmaceutical composition comprising a physiologically acceptable organic and/or inorganic vanadium compound or complex such as BMOV is for use in the manufacture of a medicament.

In one embodiment, the pharmaceutical composition comprising a physiologically acceptable organic and/or inorganic vanadium compound or complex such as BMOV is for use in the manufacture of a medicament, wherein the medicament is for use in the prevention or reduction of stress-induced metabolic derangement in a patient subjected to stress which stress elicits a state of physiological strain in the patient.

A further aspect of the invention relates to a method of prevention or amelioration of stress-induced metabolic derangement in a patient subjected to stress which stress elicits a state of physiological strain in the patient, wherein the pharmaceutical composition comprising a physiologically acceptable organic and/or inorganic vanadium compound or complex such as BMOV of the invention is administered to a patient in need thereof.

An aspect of the invention relates to the use of the pharmaceutical composition comprising a physiologically acceptable organic and/or inorganic vanadium compound or complex such as BMOV of the invention for the prevention or amelioration of stress-induced metabolic derangement in a patient subjected to stress which stress elicits a state of physiological strain in the patient.

An aspect of the invention relates to a method of prevention or amelioration of stress-induced metabolic derangement in a patient subjected to stress which stress elicits a state of physiological strain in the patient comprising administering the pharmaceutical composition comprising a physiologically acceptable organic and/or inorganic vanadium compound or complex such as BMOV of the invention to the patient.

An aspect of the invention relates to the administration of the pharmaceutical composition comprising a physiologically acceptable organic and/or inorganic vanadium compound or complex such as BMOV of the invention for prevention or amelioration of stress-induced metabolic derangement in a patient subjected to stress which stress elicits a state of physiological strain in the patient.

An aspect of the invention is the pharmaceutical composition comprising a physiologically acceptable organic and/or inorganic vanadium compound or complex such as BMOV of the invention for prevention or amelioration of stress-induced metabolic derangement in a patient subjected to stress which stress elicits a state of physiological strain in the patient.

A second aspect of the invention relates to a kit of parts comprising a container comprising at least two doses of the pharmaceutical composition comprising a physiologically acceptable organic and/or inorganic vanadium compound or complex such as BMOV for use according to the invention, the kit further comprising instructions for use of the pharmaceutical composition comprising a physiologically acceptable organic and/or inorganic vanadium compound or complex such as BMOV provided in said containers, in the prevention or reduction of stress-induced metabolic derangement in a patient who is to be subjected to stress which stress elicits a state of physiological strain in the patient.

In one embodiment, the kit of parts according to the invention is a kit comprising a first container containing a single dose of the pharmaceutical composition comprising a physiologically acceptable organic and/or inorganic vanadium compound or complex such as BMOV for use according to the invention, and a second container containing a single dose of the pharmaceutical composition comprising a physiologically acceptable organic and/or inorganic vanadium compound or complex such as BMOV for use according to the invention. For example, the kit comprises a screw-capped container comprising the pharmaceutical composition comprising a physiologically acceptable organic and/or inorganic vanadium compound or complex such as BMOV for use according to the invention as a plethora of tablets or pills for oral administration. Alternatively, the kit comprises at least two vials, each vial comprising a single dose of the pharmaceutical composition comprising a physiologically acceptable organic and/or inorganic vanadium compound or complex such as BMOV for use according to the invention, the pharmaceutical composition provided as an aqueous solution for intravenous administration, or the pharmaceutical composition provided as a powder, granules, etc., for dissolving in an aqueous solution such that an aqueous solution comprising a physiologically acceptable organic and/or inorganic vanadium compound or complex such as BMOV is provided for e.g. intravenous administration. The skilled person will appreciate that of course a manifold of alternative formulations of the pharmaceutical composition comprising a physiologically acceptable organic and/or inorganic vanadium compound or complex such as BMOV for use according to the invention can be provided, each formulation tailored towards the intended route of administration, the intended dosage regimen, the intended dose, etc., etc.

An additional aspect of the present invention is a drug delivery device or drug delivery kit, which includes an insulin-mimetic vanadium compound of the invention and a pharmaceutically acceptable carrier, wherein the device or kit is adapted for localized administration of the vanadium compound to a patient in need thereof. Localized delivery of vanadium-based insulin mimetics significantly enhanced biomechanical properties in four weeks, and the outcome is dosage dependent, with lower dosages of the vanadium compound giving superior results.

The invention is further illustrated by the following examples, which should not be interpreted as limiting the present invention in any way.

### EXAMPLES

### Example 1

The Effects of BMOV on Stress-Induced Hyperglycemia in Normal Healthy Rats—an In Vivo Assessment of the Capacity of BMOV to Prevent or Reduce Hyperglycemia.

A stress induced hyperglycemia model is developed, which model does not include any surgical procedure for inducing hyperglycemia in subjects, and which model does not require administration of any composition by infusion in order to induce hyperglycemia in subjects. The stress is elicited by infusion of adrenaline into the subject such that the subject is subjected to stress in a time controlled manner.

Aims: The objective of the exemplifying study was to show the beneficial effects of BMOV on stress- and/or trauma-induced hyperglycemia in healthy subjects.

Methods: Male Wistar rats (body weight 300-400 g) were included in the studies.

1) Series 1: Rats (n=13; 4-5/group) were infused with adrenaline at a rate of 0.1, 0.3 or 1 μg/kg/min for 180 min through the jugular vein in order to determine the optimal blood glucose level-increasing dose of adrenaline. Blood glucose levels were measured from samples taken from the femoral artery before starting the adrenaline infusion and at 15, 30, 45, 60, 90, 120, 150, 180 min after starting the adrenaline infusion.

2) Series 2: Animals (n=4; 1-2/group) were infused with adrenaline (1 μg/kg/min) or saline (1 ml/kg) for 200 min, and BMOV (0.2, 0.6, 1.7, and 5 ml/kg) or vehicle infusion for 20-20 min starting 120 min after the onset of adrenaline or saline through the right and left jugular veins, respectively. Glucose levels were measured at t=0, 120, 140, 160, 180 and 200 min.

Results: Adrenaline infusion induced a dose-dependent continuous increase in blood glucose level after 45 min. The highest dose (1 μg/kg/min) resulted in the most prominent increase with a peak level at around 120 min, which remained approximately steady until 180 min after the start of the infusion. BMOV significantly decreased hyperglycemia at 160-180 min compared to the adrenaline+vehicle control group, and a decline in the glucose levels was observed in both groups after 160 min.

Conclusions from these exemplifying in vivo experiments: BMOV significantly decreased hyperglycemia induced by high dose (1 μg/kg/min) adrenaline at 160-180 min in subjects compared to the group of subjects receiving vehicle. The infusion of adrenaline reflects the exposure of a subject to stress, such as the stress subjected to a subject's body during e.g. trauma, such as trauma inflicted during surgery.

General Test System Parameters

Experimental Animals

Altogether 31 healthy male Wistar rats weighing 300-400 g were purchased from Toxicoop Hungary Zrt. and were included in the study.

Husbandry

Housing: The animals were housed in cages that conform to the size recommendations in the most recent Guide for the Care and Use of Laboratory Animals DHEW (NIH) and EU Guidelines 63/2010. Litter material placed beneath the cage was changed at least three times/week. The animal room was temperature controlled, had a 12-hour light/dark cycle with lights on at 7 am to 7 pm, and was kept clean and vermin free.

Acclimation: The animals were conditioned to the housing facilities for at least 3 days prior to testing.

Food: Standard rodent chow ad-libitum, fasting started 12 hours prior experiment.

Water: Filtered tap water ad-libitum.

Contaminants: There are no known contaminants reasonably expected to be found in the food or water at levels which would interfere with the results of this study.

Identification: Each animal was given a sequential ID number.

Experimental Group

Series 1—Adrenaline dose determination, optimization of the paradigm

Experimental model (4-5 rats/group)

TABLE 1
Experimental setup for series 1.

| | Adrenaline infusion | Sampling | |
|---|---|---|---|
| | continuous for | (glucose | Number of |
| | 180 min | measurement) | animals |
| Group LA | Low dose | 0, 15, 30, 45, | 5 |
| (LA-1-4, -13) | 0.1 μg/kg/min | 60, 90, 120, | |
| Group MA | Medium dose | 150, 180 min | 4 |
| (MA-5-8) | 0.3 μg/kg/min | | |
| Group HA | High dose | | 4 |
| (HA-9-12) | 1.0 μg/kg/min | | |

Series 2—Control point (1-2 rats/group)

TABLE 2
Experimental setup for series 2.

| | | Treatment | | |
|---|---|---|---|---|
| | | cumulative | | |
| | | infusion of | Sampling | |
| | | 4 dose levels | (glucose | |
| | | of BMOV | measurements | |
| | Infusion for | (1.7 mg/ml); | in duplicate, | Number of |
| | 200 min | 20 min each | plasma) | Animals |
| Group 1 | Adrenaline | Vehicle only | 0, 120, 140, | 1 |
| (G1.14) | | | 160, 180, | |
| Group 2 | Adrenaline | BMOV 4x | 200 min | 2 |
| (G2.20-2.21) | | cumulative | | |
| Group 3 | Saline | BMOV 4x | | 1 |
| (G3.26) | | cumulative | | |

Methods

The term “saline” has its normal scientific meaning, and throughout the application refers to a aqueous sodium chloride (NaCl) solution with 9.0 gram NaCl per liter of water. Saline is also referred to as “saline solution”, “normal saline” (NSS, NS or N/S), “physiological saline” and “isotonic saline”.

Experimental Model for Determination of the Optimal Adrenaline Infusion Rate

In the first part of the study (Table 1, Table 3, series 1), thirteen (13) healthy male Wistar rats were anesthetized by intraperitoneal (ip.) injection of thiopental (Tiobarbital 0.5 g, Braun Medical SA) after 12 hours of fasting. After development of anesthesia, the right jugular vein was cannulated for administration of adrenaline (Tonogen 1 mg/ml injection, Gedeon Richter Plc., Budapest, Hungary; epinephrine) as a slow continuous infusion with a rate of 0.1, 0.3, or 1 μg/kg/min (after Tonogen dilutions of 50×, 16.6× and 5×, infusion volumes will be calculated on the basis of the body weights of the animals: approximately 100 μl/h), respectively for 180 min. Blood samples were collected via femoral artery cannulation before and at 15, 30, 45, 60, 90, 120, 150, 180 min after the start of adrenaline infusion via a 8-10 cm-long PESO (ID=0.4 mm; dead space 10-13 μL) cannula by directly dripping onto DCont TREND test strips (77 Elektronika) in order to determine blood glucose levels.

Experimental Model for Investigation of the Effects of BMOV Treatment on Blood Glucose Levels

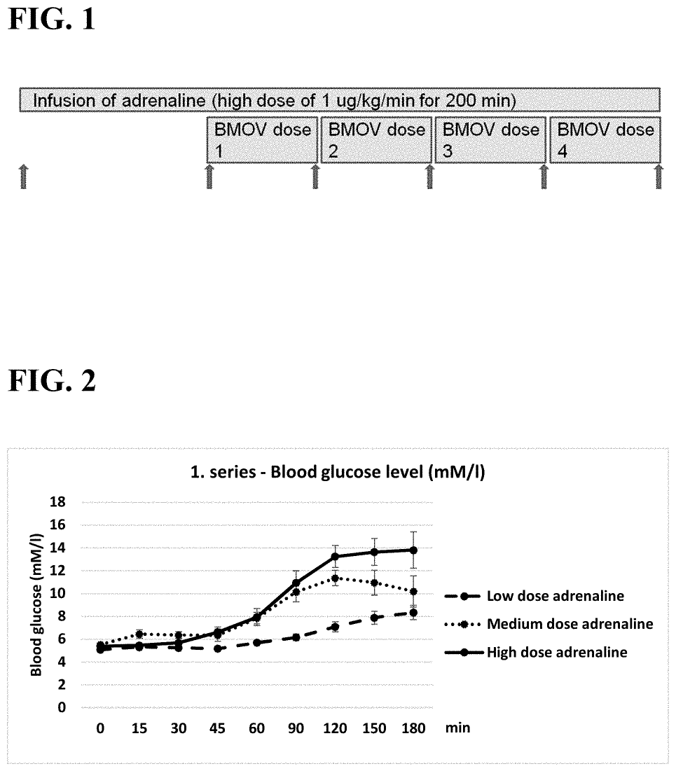

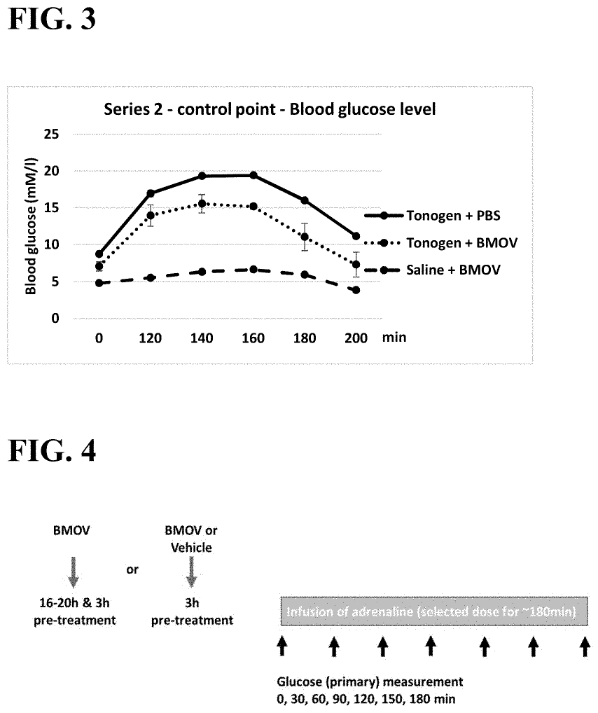

In series 2 (See Table 2), based on the results of the blood glucose level-increasing effect of adrenaline infusion, 4 healthy male Wistar rats were anesthetized as described above and the right jugular vein was cannulated for infusion of adrenaline (1 μg/kg/min of 5× dilution) or saline (1 ml/kg) for 200 min and the left jugular vein was cannulated for the infusion of the test compound BMOV (1.7 mg/ml stock solution; 0.2, 0.6, 1.7 and 5 ml/kg for 20-20 min in each animal started 120 min after the onset of adrenaline or saline infusion) or vehicle. Blood glucose levels were measured at each time point in duplicates by directly dripping blood onto DCont TREND test strips (77 Elektronika, Budapest, Hungary). Blood samples (500 μl per time) were collected via the right femoral artery into 1-mL K2EDTA-containing tubes (VACUETTE® TUBE, Greiner Bio-One Hungary Kft., Mosonmagyaróvár, Hungary) before the start of the adrenaline infusion, before and after the administration of each doses of BMOV in every 20 min (FIG. 1, FIG. 3), for measurements of glucose level. Blood samples were centrifuged at 4.000 rpm at 4° C. for 15 min. The prepared, separated samples derived from the centrifuged plasma (total volume 200 μl) were kept at −70° C. until shipment.

TABLE 3
BMOV administration in the control point of the study. Vehicle
mimicked change in infusion rate. Fresh BMOV stock solution
of 1.7 mg/ml was prepared at each experimental day.

| | mg/kg | ml/kg |
|---|---|---|
| BMOV 1 | 0.34 | 0.2 |
| BMOV 2 | 1.02 | 0.6 |
| BMOV 3 | 2.89 | 1.7 |
| BMOV 4 | 8.50 | 5.0 |

Results

In FIG. 2, the results are depicted of the above outlined experiment designated ‘1. Series, Series 1’. It is evident that intravenous introduction of adrenaline to the subjects inflicts hyperglycemic state in an adrenaline dose dependent manner, wherein the extent of the induced increase in the glucose level in the blood of the subjects increases in time during the time course in which the adrenaline is administered.

In FIG. 3, the results are shown of the treatment of the subjects in experiment ‘Series 2’ with BMOV during the course of the administration of the stress, i.e. intraperitoneal infusion of adrenaline, wherein the administration of BMOV is started with a delay with regard to the start of eliciting the stress in the subject. Administering BMOV after the start of the induction of stress and during the continued progression of the stress lowers the extent of the increase of the glucose level in the subjects.

Conclusions of Experiments ‘Series 1 and Series 2’

Series 1

Adrenaline infusion induced a dose-dependent continuous increase in blood glucose level 45 min after starting the infusion (FIG. 1, FIG. 2, Table 4, Table 5). High dose (1 μg/kg/min of 5× dilution of Tonogen epinephrine) resulted in the most prominent increase with a peak level of glucose at around 120 min after starting the infusion with the adrenaline, which remained approximately steady until the end of the administration (at 180 min). Based on these results the investigation of the effect of the test compound BMOV on blood glucose level was optimal to be tested after 120 min of the start of the adrenaline infusion in the subjects.

Series 2

The blood glucose levels start to decline after 160 min of the start of the adrenaline infusion in the subjects in both the [Tonogen epinephrine+Vehicle] test group and the [Tonogen+BMOV] group, which, without wishing to be bound to any theory, is due to the diluting effect of the relatively higher infusion volumes administered from this time point and onwards. The experimental protocol was therefore modified according to series 3. See FIG. 3 and Table 6, Table 7.

Blood Glucose Levels at Series 1

TABLE 4
Blood glucose levels of animals receiving low adrenaline dose (LA) of 0.1 μg/kg/min;
dilution of 50X for 180 min; medium adrenaline dose (MA) of 0.3 μg/kg/min;
dilution of 16.6X for 180 min and high adrenaline dose (HA) of 1 μg/kg/min;
dilution of 5X for 180 min. LA-4 died most probably due to respiratory failure.

| | Blood glucose levels (mM/l) | | | | | | | | |
|---|---|---|---|---|---|---|---|---|---|
| | 0 min | 15 min | 30 min | 45 min | 60 min | 90 min | 120 min | 150 min | 180 min |
| LA-1 | 5.5 | 5.9 | 5.5 | 5.4 | 5.3 | 5.2 | 5.4 | 5.2 | 5.9 |
| LA-1 | 5.2 | 5.8 | 5.4 | 4.3 | 5.2 | 5.2 | 5.0 | 5.5 | 5.7 |
| LA-2 | 4.7 | 4.6 | 4.6 | 5.0 | 6.0 | 6.7 | 8.1 | 8.5 | 8.4 |
| LA-2 | 4.7 | 4.6 | 4.6 | 5.2 | 6.1 | 6.5 | 8.4 | 8.5 | 8.3 |
| LA-3 | 4.5 | 5.0 | 4.8 | 4.8 | 5.3 | 5.8 | 7.2 | 9.3 | 10.4 |
| LA-3 | 4.7 | 4.9 | 4.9 | 4.9 | 5.5 | 5.7 | 7.2 | 9.2 | 10.5 |
| LA-4 | 5.3 | 5.6 | 5.8 | 5.3 | — | — | — | — | — |
| LA-4 | 5.3 | 5.7 | 5.8 | 5.3 | — | — | — | — | — |
| LA-13 | 5.5 | 5.5 | 5.6 | 5.8 | 6.0 | 7.3 | 7.5 | 8.3 | 8.8 |
| LA-13 | 5.5 | 5.5 | 5.6 | 5.8 | 6.2 | 6.9 | 7.9 | 8.6 | 8.7 |
| MA-5 | 5.3 | 5.9 | 6.0 | 4.7 | 6.4 | 6.0 | 8.7 | 7.5 | 5.3 |
| MA-5 | 5.0 | 5.7 | 5.9 | 4.3 | 6.4 | 7.4 | 8.9 | 7.1 | 5.3 |
| MA-6 | 5.5 | 6.4 | 6.0 | 5.7 | 6.7 | 9.5 | 11.7 | 12.8 | 13.2 |
| MA-6 | 5.2 | 6.3 | 5.9 | 5.8 | 6.7 | 10.1 | 11.5 | 13.0 | 13.0 |
| MA-7 | 5.5 | 7.8 | 7.8 | 8.4 | 9.9 | 11.0 | 11.1 | 9.1 | 8.0 |
| MA-7 | 5.9 | 8.3 | 7.7 | 8.3 | 9.3 | 11.5 | 11.5 | 9.2 | 8.3 |
| MA-8 | 6.0 | 5.7 | 5.8 | 6.7 | 8.6 | 12.9 | 13.6 | 14.5 | 14.2 |
| MA-8 | 5.8 | 5.5 | 5.8 | 6.8 | 8.8 | 12.8 | 13.9 | 14.5 | 14.2 |
| HA-9 | 6.5 | 5.8 | 5.5 | 5.6 | 5.7 | 6.4 | 9.8 | 9.7 | 7.5 |
| HA-9 | 6.2 | 5.4 | 5.7 | 5.6 | 5.9 | 6.3 | 9.5 | 10.2 | 7.7 |
| HA-10 | 5.7 | 5.4 | 5.3 | 5.2 | 6.4 | 12.1 | 14.5 | 15.1 | 18.3 |
| HA-10 | 5.6 | 5.4 | 5.4 | 5.4 | 6.3 | 12.3 | 14.6 | 17.1 | 18.4 |
| HA-11 | 4.3 | 5.5 | 5.7 | 7.5 | 8.4 | 11.5 | 12.5 | 11.3 | 12.3 |
| HA-11 | 4.6 | 5.1 | 5.8 | 7.8 | 9.3 | 12.0 | 12.0 | 11.2 | 12.5 |
| HA-12 | 4.9 | 5.5 | 6.2 | 7.9 | 10.5 | 12.8 | 16.6 | 17.3 | 17.0 |
| HA-12 | 5.3 | 5.5 | 5.9 | 8.0 | 11.0 | 14.2 | 16.5 | 17.3 | 16.8 |

TABLE 5
Mean and SEM values of blood glucose levels in series 1.

| | | Blood glucose level (mM/l) | | | | | | | | |
|---|---|---|---|---|---|---|---|---|---|---|
| | | Time (min) | | | | | | | | |
| | | 0 | 15 | 30 | 45 | 60 | 90 | 120 | 150 | 180 |
| Mean | Low dose | 5.09 | 5.31 | 5.26 | 5.18 | 5.70 | 6.16 | 7.09 | 7.89 | 8.34 |
| | Medium dose | 5.53 | 6.45 | 6.36 | 6.34 | 7.85 | 10.15 | 11.36 | 10.96 | 10.19 |
| | High dose | 5.39 | 5.46 | 5.69 | 6.62 | 7.93 | 10.95 | 13.25 | 13.65 | 13.81 |
| SEM | Low dose | 0.12 | 0.16 | 0.16 | 0.14 | 0.15 | 0.28 | 0.449 | 0.57 | 0.63 |
| | Medium dose | 0.13 | 0.37 | 0.30 | 0.53 | 0.51 | 0.87 | 0.67 | 1.09 | 1.37 |
| | High dose | 0.27 | 0.07 | 0.10 | 0.45 | 0.76 | 1.04 | 0.98 | 1.19 | 1.59 |

Blood Glucose Levels at Series 2

TABLE 6
Blood glucose levels in series 2.

| | | Animal | Blood glucose level (mM/l) | | | | | |
|---|---|---|---|---|---|---|---|---|
| Date | Group | ID | 0 min | 120 min | 140 min | 160 min | 180 min | 200 min |
| day n | Tonogen + | G1.14 | 8.7 | 16.5 | 19.1 | 19.3 | 16.0 | 11.1 |
| | PBS | | 8.7 | 17.4 | 19.5 | 19.5 | 16.0 | 11.2 |
| day n | Tonogen + | G2.20 | 6.0 | 11.7 | 13.5 | 15.7 | 14.2 | 10.1 |
| | BMOV | | 6.0 | 11.2 | 13.3 | 15.9 | 14.2 | 10.3 |
| day n + 1 | Tonogen + | G2.21 | 8.4 | 16.4 | 17.9 | 14.6 | 7.8 | 4.3 |
| | BMOV | | 7.8 | 16.5 | 17.5 | 14.5 | 7.9 | 4.4 |
| day n + 1 | Saline + | G3.26 | 4.7 | 5.7 | 6.3 | 6.5 | 5.9 | 3.8 |
| | BMOV | | 4.8 | 5.3 | 6.3 | 6.7 | 5.9 | 3.8 |

TABLE 7
Mean and SEM values of blood glucose levels in series 2.

| | | Blood glucose level (mM/l) | | | | | |
|---|---|---|---|---|---|---|---|
| | | Time (min) | | | | | |
| | | 0 | 120 | 140 | 160 | 180 | 200 |
| Mean | Tonogen + | 8.70 | 16.95 | 19.30 | 19.40 | 16.00 | 11.15 |
| | PBS | | | | | | |
| | Tonogen + | 7.05 | 13.95 | 15.55 | 15.18 | 11.03 | 7.28 |
| | BMOV | | | | | | |
| | Saline + | 4.75 | 5.50 | 6.30 | 6.60 | 5.90 | 3.80 |
| | BMOV | | | | | | |
| SEM | Tonogen + | 0.00 | 0.45 | 0.20 | 0.10 | 0.00 | 0.05 |
| | PBS | | | | | | |
| | Tonogen + | 0.62 | 1.45 | 1.24 | 0.36 | 1.83 | 1.69 |
| | BMOV | | | | | | |
| | Saline + | 0.05 | 0.20 | 0.00 | 0.10 | 0.00 | 0.00 |
| | BMOV | | | | | | |

### Example 2

Exemplifying Experiment Showing that Pre-Treating Subjects with BMOV before Occurrence of Stress to which the Subject is Subjected/Exposed Results in Reducing and/or Preventing Hyperglycemia During the Exposure of the Subject to Stress.

Test Article

Name: Bis(maltolato)oxovanadium (BMOV)

BMOV was dissolved in phosphate buffered saline.

General Test System Parameters

Animal Requirements

Twelve healthy male Wistar rats weighing 300-400 g were purchased from the Animal House of the University of Pecs (Hungary) and were included in the exemplifying study.

Husbandry

Housing: The animals were housed in cages that were according to the size recommendations in the current Guide for the Care and Use of Laboratory Animals DHEW (NIH) and EU Guidelines 63/2010. Litter material placed beneath the cage was changed at least three times/week. The animal room was temperature controlled, having a 12-hour light/dark cycle with lights on at 7 am to 7 pm, and was kept clean and vermin free.

Acclimation: The animals were conditioned to the housing facilities for at least 3 days prior to testing.

Food: Standard rodent chow ad-libitum, fasting for 3 hours prior to start of adrenaline infusion.

Water: Filtered tap water ad-libitum.

Contaminants: There were no known contaminants reasonably expected to be found in the food or water at levels which would interfere with the results of this study.

Identification: Each animal was given a sequential ID number.

Methods

Experimental Model

Pre-Treatment

Group 1, three healthy male Wistar rats were treated 2 times with BMOV 15 mg/kg by intraperitoneal injection (ip) 16-24 hours and 3 hours prior to start of adrenaline infusion. Group 2, three healthy male Wistar rats were treated with BMOV 15 mg/kg by intraperitoneal injection (ip) 3 hours prior to start of adrenaline infusion. Group 3, three healthy male Wistar rats were treated with PBS vehicle 3 hours prior to start of adrenaline infusion, see FIG. 4.

Investigation of the Rffects of BMOV Treatment on Blood Glucose Levels Induced by Acute Adrenaline Stress

Following 3 h fasting, the rats were anesthetized by intraperitoneal (ip.) injection of thiopental (Tiobarbital 0.5 g, Braun Medical SA). After development of anesthesia, the right jugular vein was cannulated for administration of adrenaline (Tonogen 1 mg/ml injection, Gedeon Richter Plc., Budapest, Hungary; Tonogen comprises epinephrine) as a slow continuous infusion with a rate of 0.3 microgram/kg/min Tonogen (approx. 100 microliter/h), respectively for 180 min. See Table 8 and Table 9 for further details. Blood samples were collected from femoral artery (or via right carotid artery cannulation as was more feasible for the high volumes) before and 30, 60, 90, 120, 150, 180 min after the start of adrenaline infusion via a 8-10 cm-long PESO (ID=0.4 mm; dead space 10-13 microliter) cannula by directly dripping onto point-of-care strips in duplicates for measurements of glucose level at 180 min only.

TABLE 8
BMOV administration.

| | mg/kg | mL/kg |
|---|---|---|
| BMOV | 15.0 | 2.0 |

BMOV stock solution was prepared before each experiment day at 1.7 mg/mL.

Measurements of Blood Glucose Levels in Subjects of Groups 1-3

Measurement of glucose levels was performed by DCont TREND glucometer. DCont TREND test strips (77 Elektronika, http://www.regolymedical.hu/en/measuring-tools-and-surgicalinstruments/vercukormero-d-cont-trend.html), in duplicate in order to determine blood glucose levels. See Table 9 for the blood sampling regime during the course of the experiment.

TABLE 9
Experimental groups

| | | Adrenaline infusion | | |
|---|---|---|---|---|
| | | continuous | | |
| | | (0.3 microgram/ | | |
| | | kg/min | | |
| | | for 180 min) | | |
| | | Start of adrenaline | Sampling | |
| | Treatment | infusion | (glucose) | Number |
| Group 1 | 15 mg/kg BMOV | 16-24 h following | 0, 30, 60, | 3 |
| | 2x (i.e. 16-24 h | first pre-treatment | 90, 120, | |
| | and 3 h | | 150, 180 | |
| | pretreatments) | | minutes | |
| Group 2 | 15 mg/kg BMOV | 3 h following | 0, 30, 60, | 3 |
| | 3 h pretreatment | pre-treatment | 90, 120, | |
| | | | 150, 180 | |
| | | | minutes | |
| Group 3 | Vehicle | 3 h following | 0, 30, 60, | 3 |
| | | pre-treatment | 90, 120, | |
| | | | 150, 180 | |
| | | | minutes | |

Group 1-3: 7 time points 3 animals 3 different exp. groups in duplicates, altogether 126 samples for glucose measurements.

End Points of the Study

Blood glucose levels.

Aliquoted plasma samples were prepared and stored at −70° C. until shipment.

Results

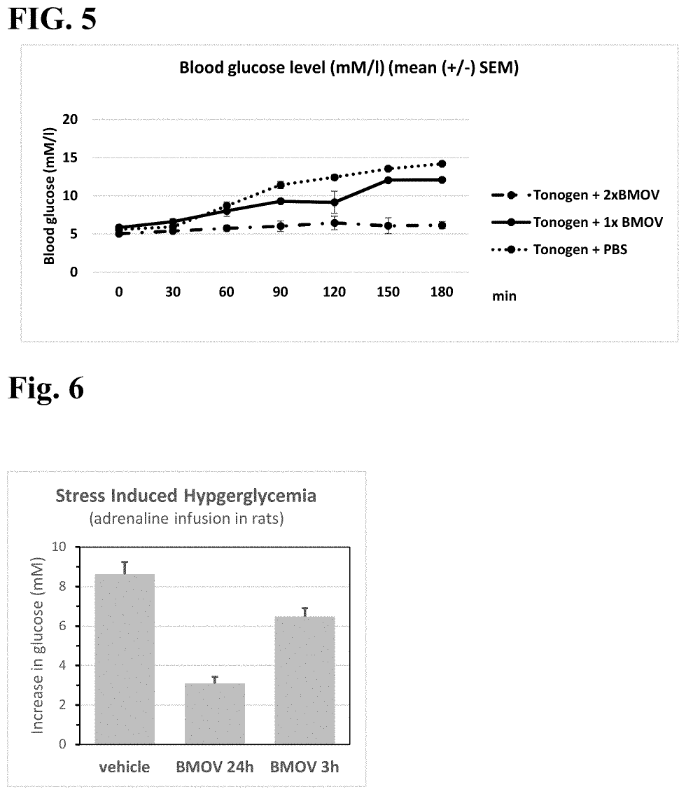

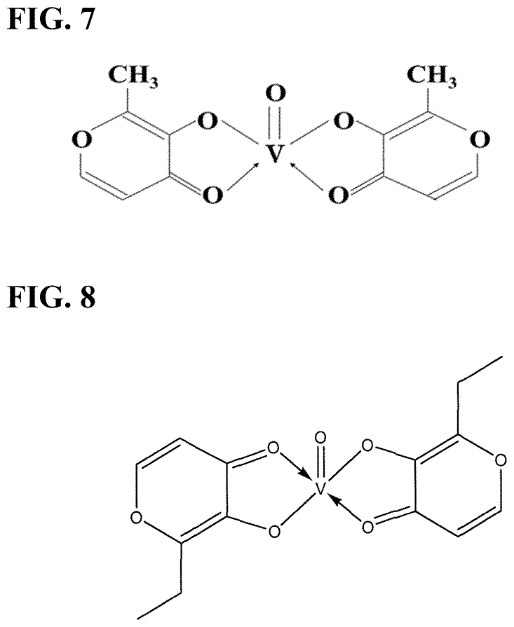

Administering a single dose of BMOV to subjects 3 h prior to inflicting stress in said subjects reduced the extent of hyperglycemia considerably during the period of stress (FIG. 5; Table 10, Table 11). Moreover, administering a first dose of BMOV to subjects 16 to 24 h prior to inflicting stress in said subjects and subsequently administering a second dose of BMOV to the subjects 3 h prior to inflicting stress in said subjects, inhibited the occurrence of hyperglycemia during the period of stress, and reduced the extent of the increase in blood glucose level to a high amount (FIG. 5; Table 10, Table 11).

The administration of two doses of a pharmaceutical composition comprising BMOV resulted in a low increase of the glucose level in the blood of the subjects subjected to adrenaline-induced stress for 180 minutes, when compared to the control group that received vehicle (phosphate buffered saline) instead of BMOV. Subjects treated twice with BMOV before stress was induced to the subjects had an increase in blood glucose level of between about −75% to about +84% during the course of 180 minutes in which stress was induced after administration of BMOV 3 h before the start of the induction of the stress. To the contrary, in the control group that was pre-treated with vehicle before stress was induced, the glucose level in the blood of the subjects increased from for example 5.7 mM/l to 13.4 mM/l at t=180 minutes and for example 5.7 mM/l at t=0 to 14.9 mM/l at t=180 minutes. See Table 10, Table 11, FIG. 5, FIG. 6. Pretreating subjects with BMOV (3 h before occurrence of stress, or at 16-24 h and subsequently at 3 h before occurrence of the stress) before occurrence of a stressful insult (here, the infusion of adrenaline starting at t=0 minutes) thus results in a surprisingly high reduction of the occurrence of hyperglycemia in the subjects suffering from stress. In FIG. 6, the glucose level in the blood of the subjects exposed to the stress is shown at the time point 180 minutes after the start of the adrenaline infusion.

TABLE 10
Blood glucose levels in subjects exposed to stress (administration of adrenaline) after
being pre-treated once (‘Tonogen + 1x BMOV’) or twice (‘Tonogen + 2x BMOV’)
with BMOV before the stress was inflicted to the subject, compared to subjects exposed to
the same stress and without being pretreated with BMOV (‘Tonogen + PBS’).

| | | Animal | Blood glucose (mM/l) | | | | | | |
|---|---|---|---|---|---|---|---|---|---|
| Date | Group | ID | 0 | 30 | 60 | 90 | 120 | 150 | 180 |
| Aug. 8, 2017 | Tonogen + 2x | G4.32 | 4.8 | 5.3 | 5.3 | 5.8 | 6.7 | 7.5 | 6.9 |
| | BMOV | | 5.2 | 5.2 | 5.2 | 5.8 | 6.7 | 7.4 | 6.7 |
| Sep. 8, 2017 | Tonogen + 2x | G4.33 | 5.4 | 4.8 | 4.4 | 3.4 | 2.7 | 1.4 | No data |
| | BMOV | | 5.2 | 5 | 4.3 | 3.2 | 2.6 | 1.3 | available - |
| | | | | | | | | | exit at |
| | | | | | | | | | 155 min |
| Oct. 8, 2017 | Tonogen + 2x | G4.34 | 4.4 | 5.3 | 6.3 | 6.4 | 7.4 | 7.7 | 7 |
| | BMOV | | 4.3 | 5 | 6.4 | 6.8 | 7.7 | 7.9 | 6.8 |
| Nov. 8, 2017 | Tonogen + 2x | G4.44 | 5.1 | 6.3 | 7.1 | 8.5 | 9.1 | 7.6 | 4.6 |
| | BMOV | | 5.8 | 6.1 | 7 | 8.2 | 8.6 | 7.7 | 4.8 |
| | | Mean | 5.025 | 5.375 | 5.75 | 6.0125 | 6.4375 | 6.0625 | 6.133333 |
| | | SD | 0.50356 | 0.539179 | 1.104536 | 1.948213 | 2.481899 | 2.912504 | 1.116542 |
| | | SEM | 0.17803 | 0.190629 | 0.390512 | 0.688797 | 0.877484 | 1.029726 | 0.455826 |
| Jul. 8, 2017 | Tonogen + 1x | G5.35 | 6.1 | 6.9 | 8.4 | 9.4 | 10.7 | 12.3 | 12.4 |
| | BMOV | | 6.7 | 6.9 | 8 | 9.2 | 11.2 | 12.6 | 12.2 |
| Sep. 8, 2017 | Tonogen + 1x | G5.36 | 5.1 | 5.1 | 6 | 9.8 | 12.2 | 11.7 | 11.9 |
| | BMOV | | 4.9 | 5.9 | 6 | 9.7 | 11.5 | 11.6 | 11.9 |
| Nov. 8, 2017 | Tonogen + 1x | G5.37 | 6.3 | 7.5 | 9.7 | 8.6 | 4.6 | No data available - | |
| | BMOV | | 5.9 | 7.2 | 10 | 9 | 4.7 | exit at 140 min | |
| | | Mean | 5.83333 | 6.583333 | 8.016667 | 9.283333 | 9.15 | 12.05 | 12.1 |
| | | SD | 0.70048 | 0.904249 | 1.734839 | 0.449073 | 3.519517 | 0.479583 | 0.244949 |
| | | SEM | 0.28597 | 0.369158 | 0.708245 | 0.183333 | 1.436837 | 0.239792 | 0.122474 |
| Jul. 8, 2017 | Tonogen + | G6.38 | 5.7 | 6.2 | 10.4 | 12.8 | 13 | 13.7 | 14.5 |
| | PBS | | 5.7 | 6 | 10 | 12.7 | 13 | 13.4 | 14.9 |
| Aug. 8, 2017 | Tonogen + | G6.39 | 4.9 | 5.6 | 8.1 | 11.1 | 12.8 | 14.2 | 14.5 |
| | PBS | | 5.5 | 5.5 | 8.2 | 11.2 | 12.9 | 14.4 | 14.4 |
| Oct. 8, 2017 | Tonogen + | G6.40 | 6 | 6.2 | 7.8 | 10.2 | 11.6 | 12.8 | 13.5 |
| | PBS | | 5.7 | 6.1 | 7.6 | 10.5 | 11.3 | 12.8 | 13.4 |
| | | Mean | 5.58333 | 5.933333 | 8.683333 | 11.41667 | 12.43333 | 13.55 | 14.2 |
| | | SD | 0.37103 | 0.307679 | 1.200694 | 1.098029 | 0.771146 | 0.680441 | 0.60663 |
| | | SEM | 0.15147 | 0.12561 | 0.490181 | 0.448268 | 0.314819 | 0.277789 | 0.247656 |

TABLE 11
Mean blood glucose levels in subjects exposed to stress (administration of adrenaline)
after being pre-treated once (‘Tonogen + 1x BMOV’) or twice (‘Tonogen +
2x BMOV’) with BMOV before the stress was inflicted to the subject, compared to subjects
exposed to the same stress and without being pretreated with BMOV (‘Tonogen + PBS’).

| | | Blood glucose (mM/l) | | | | | | |
|---|---|---|---|---|---|---|---|---|
| | | 0 | 30 | 60 | 90 | 120 | 150 | 180 |
| Mean | Tonogen + | 5.025 | 5.375 | 5.75 | 6.0125 | 6.4375 | 6.0625 | 6.133333 |
| | 2xBMOV | | | | | | | |
| | Tonogen + 1x | 5.833333333 | 6.583333 | 8.016667 | 9.283333 | 9.15 | 12.05 | 12.1 |
| | BMOV | | | | | | | |
| | Tonogen + | 5.583333333 | 5.933333 | 8.683333 | 11.41667 | 12.43333 | 13.55 | 14.2 |
| | PBS | | | | | | | |
| SEM | Tonogen + | 0.178034908 | 0.190629 | 0.390512 | 0.688797 | 0.877484 | 1.029726 | 0.455826 |
| | 2xBMOV | | | | | | | |
| | Tonogen + 1x | 0.285968141 | 0.369158 | 0.708245 | 0.183333 | 1.436837 | 0.239792 | 0.122474 |
| | BMOV | | | | | | | |
| | Tonogen + | 0.151474237 | 0.12561 | 0.490181 | 0.448268 | 0.314819 | 0.277789 | 0.247656 |
| | PBS | | | | | | | |

1. A method for the prevention or reduction of stress-induced metabolic derangement in a patient subjected to stress, the method comprising administering to the patient a pharmaceutical composition comprising a physiologically acceptable organic and/or inorganic vanadium compound or complex and further comprising a pharmaceutically acceptable excipient

2. The method of claim 1, wherein the pharmaceutical composition is administered to the patient before the patient is subjected to the stress.

3. The method of claim 1, wherein the metabolic derangement comprises hyperglycemia, preferably the metabolic derangement is hyperglycemia.

4. The method of claim 1, wherein the physiologically acceptable organic and/or inorganic vanadium compound or complex is bis(maltolato)oxidovanadium (BMOV) or bis(ethylmaltolato)oxovanadium(IV) (BEOV), preferably BMOV.

5. The method of claim 1, wherein the pharmaceutical composition comprises as the sole active pharmaceutical ingredient BMOV or BEOV, or wherein the pharmaceutical composition comprises as the active pharmaceutical ingredient a combination of BMOV and BEOV.

6. The method of claim 5, wherein the pharmaceutical composition is administered to the patient at least once at a time point between 96 h and 0 h before the patient is subjected to the stress, preferably at a time point between 72 h and 1 h, more preferably at a time point between 48 h and 2 h, most preferably at a time point between 24 h and 3 h before the patient is subjected to the stress.

7. The method of claim 1, wherein the stress is a trauma, such as a trauma caused by surgery, a trauma caused by a medical procedure, a trauma caused by iatrogenic injury, and/or wherein the stress to which the patient is subjected is caused by any one or more of an infection, administration of one or more medicine(s), either long term or short term acute mental stress due to a life threatening situation, such as a potentially life threatening situation and an accident.

8. The method of claim 1, wherein the pharmaceutical composition comprising a physiologically acceptable organic and/or inorganic vanadium compound or complex is administered orally to the patient, or administered intravenously to the patient, or administered intraperitoneally, or administered intramuscularly, or administered intradermally, preferably the pharmaceutical composition comprising is administered intravenously to the patient or intradermally or intraperitoneally.

9. The Method of claim 8, wherein the pharmaceutical composition is an aqueous solution, preferably an aqueous solution comprising between about 0,001 mg and 10 mg of a physiologically acceptable organic and/or inorganic vanadium compound or complex per ml of the pharmaceutical composition, preferably between about 0,5 mg/ml and 5 mg/ml, more preferably between about 2 mg of a physiologically acceptable organic and/or inorganic vanadium compound or complex per ml.

10. The method of claim 9, wherein the physiologically acceptable organic and/or inorganic vanadium compound or complex is BMOV or BEOV or a combination thereof, preferably BMOV.

11. The method of claim 9, wherein the at least one pharmaceutically acceptable excipient comprises phosphate buffered saline, said phosphate buffered saline comprising 1.86 g/l NaH₂PO₄. H₂O, 9.50 g/l Na₂HPO₄. 2 H₂O and 4.40 g/l NaCl, preferably said pharmaceutically acceptable excipient consisting of phosphate buffered saline consisting of about 1.86 g/l NaH₂PO₄. H₂O, about 9.50 g/l Na₂HPO₄. 2 H₂O and about 4.40 g/l NaCl.

12. The method of claim 8, wherein the pharmaceutical composition comprising a physiologically acceptable organic and/or inorganic vanadium compound or complex is administered orally to the patient and wherein the pharmaceutical composition is provided as a capsule, a tablet, a pill, or a water-miscible powder for oral administration.

13. The method of claim 1, wherein a single dose of the pharmaceutical composition comprising a physiologically acceptable organic and/or inorganic vanadium compound or complex is administered to the patient, or wherein the dosage regimen is administration of at least two doses of the pharmaceutical composition comprising a physiologically acceptable organic and/or inorganic vanadium compound or complex to the patient per between 12 h and 48 h, preferably two doses between 16 h and 24 h.

14. The method of claim 1, wherein a dose of pharmaceutical composition comprising a physiologically acceptable organic and/or inorganic vanadium compound or complex which is administered to the patient comprises between 0.01 mg and 30 mg of a physiologically acceptable organic and/or inorganic vanadium compound or complex per kg body weight of the patient, preferably between 0.1 mg and 15 mg of a physiologically acceptable organic and/or inorganic vanadium compound or complex per kg body weight of the patient, more preferably between about 0.15 mg and 10 mg of a physiologically acceptable organic and/or inorganic vanadium compound or complex per kg body weight of the patient, most preferably between about 0.5 mg and 2.5 mg of a physiologically acceptable organic and/or inorganic vanadium compound or complex per kg body weight of the patient.

15. The method of claim 1, wherein the pharmaceutical composition is administered to the patient in the time period in which the patient is subjected to the stress.

16. The method of claim 1, wherein a first single dose of the pharmaceutical composition is administered to the patient at a time point of between 96 h and 10 h, preferably between 72 h and 12 h, more preferably between 48 h and 14 h, most preferably between 24 h and 16 h before the patient is subjected to the stress and a second single dose of the pharmaceutical composition is administered to the patient at a time point of between 8 h and 0 h, preferably between 5 h and 3 h before the patient is subjected to the stress, preferably said first single dose is administered between 24 h and 16 h and said second single dose is administered about 3 h before the patient is subjected to the stress.

17. The method of claim 1, wherein a first single dose of the pharmaceutical composition is administered to the patient at a time point of between 96 h and 0 h, preferably between 72 h and 1 h, more preferably between 48 h and 2 h, most preferably between 24 h and 3 h before the patient is subjected to the stress and a second single dose of the pharmaceutical composition is administered to the patient during the period in which the patient is subjected to the stress.

18. The method of claim 1, wherein a first single dose of the pharmaceutical composition is administered to the patient at a time point of between 96 h and 10 h, preferably between 72 h and 12 h, more preferably between 48 h and 14 h, most preferably between 24 h and 16 h before the patient is subjected to the stress and a second single dose of the pharmaceutical composition is administered to the patient at a time point of between 8 h and 0 h, preferably between 5 h and 3 h before the patient is subjected to the stress and a third single dose of the pharmaceutical composition is administered to the patient during the period in which the patient is subjected to the stress.

19. The method of claim 16, wherein the pharmaceutical composition is provided as an aqueous solution or as a solid dosage form such as a powder, a tablet, a pill, and a capsule, and wherein a dose of the pharmaceutical composition comprises between about 0.01 mg and 30 mg of a physiologically acceptable organic and/or inorganic vanadium compound or complex per kg body weight of the patient, preferably about 0.5 mg to 2.5 mg of a physiologically acceptable organic and/or inorganic vanadium compound or complex per kg body weight of the patient.

20. The method of claim 14, wherein the pharmaceutical composition is provided as an aqueous solution or as a solid dosage form such as a powder, a tablet, a pill, and a capsule, and wherein a dose of the pharmaceutical composition comprises between about 0.01 mg and 30 mg of a physiologically acceptable organic and/or inorganic vanadium compound or complex per kg body weight of the patient, wherein administering a dose of the pharmaceutical composition to said patient provides for a plasma exposure level of between 25 ng/ml and 2500 ng/ml elemental vanadium, or preferably between 100 ng/ml and 1000 ng/ml in said patient.

21. The method of claim 16, wherein the stress to which the patient is subjected is a trauma, such as a trauma caused by surgery, a medical trauma caused by a medical procedure, a trauma caused by iatrogenic injury, and/or wherein the stress to which the patient is subjected is caused by any one or more of an infection, administration of one or more medicine(s), either long term or short term acute mental stress due to a life threatening situation, such as a potentially life threatening situation and an accident.

22. The method of claim 1, wherein the increase of glucose content in the blood of the patient during the period in which the patient is subjected to the stress is less than about 100%, preferably less than about 65%, more preferably less than about 30%, most preferably less than about 6%, compared to the glucose content in the blood of the patient at a time point 3 h to 0 minute before the patient is subjected to the stress, preferably compared to the glucose content in the blood of the patient at the time point at which the patient starts to be subjected to the stress.

23. The method of claim 1, wherein the increase of glucose content in the blood of the patient during the first one to eight hours, preferably during the first about three hours of the period in which the patient is subjected to the stress is less than about 100%, preferably less than about 65%, more preferably less than about 30%, most preferably less than about 6%, compared to the glucose content in the blood of the patient at a time point 3 h to 0 minute before the patient is subjected to the stress, preferably compared to the glucose content in the blood of the patient at the time point at which the patient starts to be subjected to the stress.

24. The method of claim 1, wherein the increase of glucose content in the blood of the patient during the first one to eight hours, preferably during the first about three hours of the period in which the patient is subjected to the stress is between about −25% and about 100%, preferably between about 0% and about 75%, more preferably between about 5% and about 65%, most preferably about 30%, compared to the glucose content in the blood of the patient at a time point 3 h to 0 minute before the patient is subjected to the stress, preferably compared to the glucose content in the blood of the patient at the time point at which the patient starts to be subjected to the stress.

25. The method of claim 1, wherein the stress that is subjected to the patient is stress caused by trauma occurring during any one or more of surgery such as cardiac surgery, amputation surgery, tumor resection surgery, brain surgery, transplantation surgery, surgery related to restoring hemostasis, surgery related to treating bone fracture, surgery related to organ or bone restoration such as hip transplant surgery, blood vessel surgery, bypass surgery, closing of wounds, treatment of injuries to the skin, bones, organs, tissue, arteries, veins, a medical procedure, iatrogenic injury, an infection, administration of one or more medicine(s), acute mental stress, an accident.

26. A kit of parts comprising a container comprising at least two doses of the pharmaceutical composition comprising a physiologically acceptable organic and/or inorganic vanadium compound or complex as defined in claim 1, the kit further comprising instructions for use of the pharmaceutical composition comprising the physiologically acceptable organic and/or inorganic vanadium compound or complex provided in said containers, in the prevention or reduction of stress-induced metabolic derangement in a patient who is to be subjected to stress which stress elicits a state of physiological strain in the patient.

27. (Orginal) The kit of parts according to claim 26, wherein the kit comprises a first container containing a single dose of the pharmaceutical composition comprising the physiologically acceptable organic and/or inorganic vanadium compound or complex, preferably BMOV, and a second container containing a single dose of the pharmaceutical composition comprising the physiologically acceptable organic and/or inorganic vanadium compound or complex, preferably BMOV.
